SEQUENCE LISTING (6 sequences)

SEQ ID NO: 1 - DNA, 2307 bp, Homo sapiens
cacagatccttgaaagggttttggttttgactgagcagaatggtgccattgcagatcaacacgcactttaaataatcaaaacccctatctttcccctgggaaggaggaagccgaggaaggctgtgtttctgactcacacgggggagtcgggggagtcgtaaacaaccctgaagagaacagccaggcctgttcccgcaagcccagacaatgccggtggaggaatttgtggctggctggatctctggcgctctgggcttggtcctgggacacccgtttgacactgtaaaggtgaggctgcagacccagaccacctaccggggcatcgttgattgcatggtcaagatttaccgccatgagtccctcctgggcttcttcaagggaatgagcttccccattgccagcatagctgtggtcaactctgtcctgtttggggtctatagcaacaccctgctggtgctcacggccacctcccaccaggagcggcgggcccagccgcccagctacatgcacatcttcctagcgggctgcaccggggggttcctgcaggcctactgtctggctccttttgacctcatcaaagtccggctacaaaaccagacagagccaagggcccagccagggagccccccaccccggtaccaggggcccgtgcactgtgcagcctccatcttccgggaggaggggccccgggggctgttccgaggagcctgggccctgacgctgagggacacccccacggtggggatctacttcatcacctatgaagggctctgtcgccagtacacaccagaaggccagaatcccagctcagccacggtgctggtggcagggggctttgcaggcattgcttcctgggtggcagccacgcccttagacgtgatcaagtcccggatgcagatggatggactgagacgcagagtgtaccaggggatgctggactgcatggtgagcagcatccggcaggaaggactgggagtcttcttccggggggtcaccatcaacagtgcccgcgcctttcccgtcaatgctgtcaccttcctcagctacgaatatctcctccgctggtggggatgagccctgcggcaatgccagcagctccccatcaggcccacggcctggaggccagtttgagattggaggccaggttgaaagcttgcaaatcagtgcaagaggctcagcccttcctaaccaaggtgcctcccacccgcgcagatctgggctgggcagacacctgtgggagccggaagccagggggcctgtgcagcctccctgtgtagctggccttgactcctttgcctcccacatctgtgaaacagggagcatgaggcacaagtgagctggcaagtggtgctggtgacatcccagctcctgtcctgtgccttcacctctttttttttttttttttttttttgagatggagtctttctctgtcacccaggctggaatgcagtggcgtgatctcggctcactgcaacctccgcctttccaccttccgggttcaagcaactctcctgcctcagcctcccaagtagctgggactataggcgcccgccaccacacccagctaaatttttgtatttttagtagagacagggtttcaccatgtcagccaggatggtcttgatctcctgactttgtgatccgcccgcgtcggcctctcaaagtgctgggattacaggcgtgagccaccgcgcccggctgccttcacctcttaaggagctctgagactccacttctgagagtccctgcggcctcccacctccctgcctttcaaagctctctcccccatgacccaggataaccctatgtctcctcccccagaatccttcagtggctctcatcaccttcaggaaaagcccaaactccttccaccctccaggttcctccctccccacgaggctttgttctcctggggttgcttcctggaccctgaacaagttgtgctctcattggccgggcctggccagcagtgcacagtgcctggcaggttgactctaccatccccggggctggccccgctctcctccgagacccaggctgagcccagtcccctcacccttccttgacttacctccccacctgaggctgactttggggttcccagacaccctacccacacacatgccttgatcatagcacttgcctgcgcttcttcagagtcattaatttgcttctcggcttccccactggactgtgagctgcctgaggtcagggattgcgctttggatggtttccagcctgagcctggtgcttgaacagacgtgtgcaataaatgctcgttaaatgatgaaaaaaaaaaaaaaaaaa

SEQ ID NO: 2 - protein, 288 aa, Homo Sapiens
MPVEEFVAGWISGALGLVLGHPFDTVKVRLQTQTTYRGIVDCMVKIYRHESLLGFFKGMSFPIASIAVVNSVLFGVYSNTLLVLTATSHQERRAQPPSYMHIFLAGCTGGFLQAYCLAPFDLIKVRLQNQTEPRAQPGSPPPRYQGPVHCAASIFREEGPRGLFRGAWALTLRDTPTVGIYFITYEGLCRQYTPEGQNPSSATVLVAGGFAGIASWVAATPLDVIKSRMQMDGLRRRVYQGMLDCMVSSIRQEGLGVFFRGVTINSARAFPVNAVTFLSYEYLLRWWG

SEQ ID NO: 3 - DNA, 9645 bp, Homo sapiens
ggtttgtaatgatagggcggcagcagcagcagcagcagcagtggtggaacgaggaggtggagaattgagagcacgatgcatacacaggtgtttctgagtagtaattagatcgctgtgaaggaaaaagcacacctttgagttttcacctgtgaacactatagcgctgagagagacagtctgaaagcagaggaagacatcgatcagtaacaccaagagacaccaaagttgaaagttttgttttctttccctctgttttatttttcccccgtgtgtccctactatggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcggagaaaataaccagaacattacaaataatactggaagttgtaccagaagaagataagttatgaactaatggacttgagatcttggcaatctgcccaaggggagacacaaaatagggatttttacttcattttctgaaagtctagagaattacaactttggtgataaacaaaaggagtcagttatttttattcatatattttagcatattcgaactaatttctaagaaatttagttataactctatgtagttatagaaagtgaatatgcagttattctatgagtcgcacaattcttgagtctctgatacctacctattggggttaggagaaaagactagacaattactatgtggtcattctctacaacatatgttagcacggcaaagaaccttcaaattgaagactgagatttttctgtatatatgggttttgtaaagatggttttacacactatagatgtctatactgtgaaaagtgttttcaattctgaaaaaaagcatacatcatgattatggcaaagaggagagaaagaaatttattttacattgacattgcattgcttccccttagataccaatttagataacaaacactcatgctttaatggattatacccagagcactttgaacaaaggtcagtggggattgttgaatacattaaagaagagtttctaggggctactgtttatgagacacatccaggagttatgtttaagtaaaaatccttgagaatttattatgtcagatgttttttcattcattatcaggaagttttagttatctgtcatttttttttttcacatcagtttgatcaggaaagtgtataacacatcttagagcaagagttagtttggtattaaatcctcattagaacaaccacctgtttcactaataacttacccctgatgagtctatctaaacatatgcattttaagccttcaaattacattatcaacatgagagaaatcaccaacaaagaagatgttcaaaataatagtcccatatctgtaatcatatctacatgcaatgttagtaattctgaagttttttaaatttatggctatttttacacgatgatgaattttgacagtttgtgcattttctttatacattttatattcttctgttaaaatatctcttcagatgaaactgtccagattaattaggaaaaggcatatattaacataaaaattgcaaaagaaatgtcgctgtaaataagatttacaactgatgtttctagaaaatttccacttctatatctaggctttgtcagtaatttccacaccttaattatcattcaacttgcaaaagagacaactgataagaagaaaattgaaatgagaatctgtggataagtgtttgtgttcagaagatgttgttttgccagtattagaaaatactgtgagccgggcatggtggcttacatctgtaatcccagcactttgggaggctgagggggtggatcacctgaggtcgggagttctagaccagcctgaccaacatggagaaaccccatctctactaaaaatacaaaattagctgggcatggtggcacatgctggtaatctcagctattgaggaggctgaggcaggagaattgcttgaacccgggaggcggaggttgcagtgagccaagattgcaccactgtactccagcctgggtgacaaagtcagactccatctccaaaaaaaaaagattatatatatatatatatgtgtgtgtatgtgtgtgtgtgtgtgtgtgtgtatatatatatatatatatatacacacacacacacacactttttatatatatatatatatatatatagtggaacttacaaatgagagtaatataatgatgaaattttgaactgttatttataaacatctaaggtaaaatggttagtcatggccagagtatgtttcatcctttaatttttgtccatttgaaaataaggatttttgaaagaattataccaattaaaattattaaaggcaaacatagaattcataaaaaattgtccaaagtagaaatgatgacctataatttggagcatttccaattcagtaatttcaattttgctcttgaaaacatttaatatatatccaagactgacatttctttagctgaacctaacgtttgggtctctgagtgaatttataataactccttccttccttagcatagggttttcaaaatttgatttataattcctatttccagtaaatattgttcatttgtccacatctctccctatgatatgttgctggaggtaagaatttctttcatattcctattttttttttccccatagactaggctcatagaatttaaacaagcaaattttcctgagctttttcttgccaaatgaaagaagactggtaaattctcatagagaggtttgtgtagttcttggctcttcctggggttaatgtgcttatattcacagtggcaaattggtctcagactttaatttatttatttttgatttgaatttctctttaaaagtatcaatttaaaaggtaactagaattattctttctcattttcaaaagtgatttttgcattattaaatttccctgccattgtaatgccatttcacgcagaaaaaaagtcagccagtaattaagaaaaaaagtgatggagattaagtagtattttggcttatttttaggactcatcatgagaagacacagttcctttaatcaggaaattaatatccataattttcactcaaaattgcagtatgtaaagcagattctcaaaaactctcctgaacacttatttatatatatgtttttatataagtaaaatttttctcatatttttatacgatatgcacacacacacatacatgcacatactacttactacatgttctgtacttgtactttgtaccatgcatattcaaatgtttatatacataagtttattataacataaacagtaaaagtaatgaatactgtttaaaataactaatatagtattttttaatttttgtggggatggattctcaaatacttgtgattttaaaagattctaaagctaaaacacaacttgattttaaaaagaatgattctccttacacaattataaatatttgcagtaaatattttccttataatactgttttgaccccatttaaaaagtattagattatattcctttgatccaatgaaaactgaaccttataaatggttagctgaaagtagaccttattcttgtccttctttagaagagtaaagatttgtcctagggaagatggctgacttcggttcccaacatgcgtatgcatttagactgtagctcctcagccctgtggacacaaaatttggacagcttattaggttacgttagcaatgcatgacggtttctccaacactaagatattcacgttgaaacagatttcctgttcgtcttatgtgtctggtaaaattgtttccccaattacaatttgacatatcaatagagggttaacaagagtataattacataacagaattcctcatgaactgtaatcagtctacaggaaaatcattattttatcttgatttgcagatgaatatactgctaagaaagggagcaactctgacctttgttaaagttgatcttttgtaattgaggtataaggtatgaaaagataaaaaaccgaaggccagagaatcaggaaatgaaagatagtatggactgaaggtaacaatattttaatgttatgcaatatagtcagagaaatattaaaaattagttgtttgctgtgcataggtggatctcgcaggaagctaatgaaacctaagcttcagtgcctctcacttagacatgttccattcgaggtcctgaacctaactttgtattaggaattctgtactaattttgttgaagaagaccagcaaagttgtgtacacttctacccccacaaaatctgcattgtccatgtgagtaaagtaaaataattcctgttatttttttctgttagaaataagtatggaggatatgtttttaaaaatttatgagttaattgaaatatccatatataacaagtgactttctcacaatatatatgatgtgatatatagggagatagtttcactttcatcatattttatacgttgattctgaactatagaaaaataataaatgggattttaattatagctcttagttgggaaagaaatatagagagatgtgggatttgaatgcccatgaaagacattttattttacttgaatatattcttgcttcactttaccctccataatatgttgtacattagtgctgatcaagtttacagagttacattttgctttcctaaccattcagtcaggaattaaaatatggcattgtataacaactgggaagaagctcatagtggatataaattagagtagataatgggtcaccttgatagcctctgtttacattacttgtatatgggcaaaataattattacctatacgtgtatttaagcttaattttcatataaacagtatttttaatctatgttaaaatagataatatctaaaagtgtgatctctaggtagtccttagtttattagtactgtacttcaaaaagatttttaaataggtccggcacggtggctcatgcctgtaatcccagcactttgggaggctgaggcgggcgaatcacctgaggtcaggagttcgagatcagcctggccaacatggtgaaaccctgtctcaactaaaaatataaaaattagccgggcgtggtggcaggcgcctgtaatcccagctactcgggaggctgaggcaggagaatcacttgaacccaaggggcagaagctgcagttagccaagatcgcatcattgcactccagcctaggggacaagagcgcgagacttcatctcaaaaaaaaaaaaaaaaaaaaaaaaagatttttaaataatagctaaaggtatgctctctaggtcatccttagtttattagtactgtacttaaaaattattttttaatagtcaattttgggagataattatttctttccttatattttccaattagttggtgtctaaaaataaatgttttgtctaattttagatcaggtatacattcacaaaagcataaatcatagtctcacaggaaattcaccaattttccatatgtcgtgagataactgtcctttctacaacctcataacaatgaatttatataattacctagattttcttagtgtgaatctacccattagttttattttcttggtagttatttttttccctcctctctgttactattggccttaaaatacacagaggacggttacagtgtcctaatagctgttacatgtgtgtgtttcagcgtacttgaatcaagtgtacatttatagtaccaataaccgcctttacagctttacagttaacaattctctcacaaaactgtagagcattaggcatctgagagccatagagggccaactttgttccagagtgaacatgctttttttcctcaacatatacactactgattttttttaaaagtatgactttcaagtgaattaatgtattggttaggagaactgcttgctaagtccttattacctcttgttaaagcctcagaaggccgtgctgaaagccagaggggaaaaaaagagtaatgcacaggtatctcttttgcagtggtgactgtattttgagtaccttgtgtgacagggtattattacagcatcttgtgggaaaacctattaggcctttgcatgttaaagctgtataatttgttgggttgtgagtggtctgacttaaatgtgtattataaaatttagacatcaaattttcctactaactaactttattagatgcatacttggaagcacagtcatatcacactgggaggcaatgcaatgtggttacctggtcctaggtttgaactgtcttatttcaaaagatttctgaattaatttttccctagaatttctccttcattccaaagtacaaacatactttgaagaatgaaacagattgttcccatgaatgtatgctcatactcgactagaaacgatctatgttaaatgactgtgtatatgaattatttcaagtactaccccaaataactttcttattgctctgaaagaagaaaagcaatgtaaatcactatgattattgcacaaacaaccagaattctccaacaattttaagtaatctgatcctcttcttggagaaaattgttacctaatagtttttccttatgaatgttattactactggtataaatcaaatttctataaatttcctacttaagtcttaagaactgggttcttcctttgatgttattcatgttcagaaaggaaacaacactttactcttttaggacaattcctagaatctatagtagtatcaggatatattttgctttaaaatatattttggttattttgaatacagacattggctccaaattttcatctttgcacaatagtatgacttttcactagaacttctcaacatttgggaactttgcaaatatgagcatcatatgtgttaaggctgtatcatttaatgctatgagatacattgttttctccctatgccaaacaggtgaacaaacgtagttgttttttactgatactaaatgttggctacctgtgattttatagtatgcacatgtcagaaaaaggcaagacaaatggcctcttgtactgaatacttcggcaaacttattgggtcttcattttctgacagacaggatttgactcaatatttgtagagcttgcgtagaatggattacatggtagtgatgcactggtagaaatggtttttagttattgactcagaattcatctcaggatgaatcttttatgtctttttattgtaagcatatctgaatttactttataaagatggttttagaaagctttgtctaaaaatttggcctaggaatggtaacttcattttcagttgccaaggggtagaaaaataatatgtgtgttgttatgtttatgttaacatattattaggtactatctatgaatgtatttaaatatttttcatattctgtgacaagcatttataatttgcaacaagtggagtccatttagcccagtgggaaagtcttggaactcaggttacccttgaaggatatgctggcagccatctctttgatctgtgcttaaactgtaatttatagaccagctaaatccctaacttggatctggaatgcattagttatgaccttgtaccattcccagaatttcaggggcatcgtgggtttggtctagtgattgaaaacacaagaacagagagatccagctgaaaaagagtgatcctcaatatcctaactaactggtcctcaactcaagcagagtttcttcactctggcactgtgatcatgaaacttagtagaggggattgtgtgtattttatacaaatttaatacaatgtcttacattgataaaattcttaaagagcaaaactgcattttatttctgcatccacattccaatcatattagaactaagatatttatctatgaagatataaatggtgcagagagactttcatctgtggattgcgttgtttcttagggttcctagcactgatgcctgcacaagcatgtgatatgtgaaataaaatggattcttctatagctaaatgagttccctctggggagagttctggtactgcaatcacaatgccagatggtgtttatgggctatttgtgtaagtaagtggtaagatgctatgaagtaagtgtgtttgttttcatcttatggaaactcttgatgcatgtgcttttgtatggaataaattttggtgcaatatgatgtcattcaactttgcattgaattgaattttggttgtatttatatgtattatacctgtcacgcttctagttgcttcaaccattttataaccatttttgtacatattttacttgaaaatattttaaatggaaatttaaataaacatttgatagtttacataa

SEQ ID NO: 4 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 5 - DNA, 1317 bp, Homo sapiens
cttcctgacccaggggctccgctggctgcggtcgcctgggagctgccgccagggccaggaggggagcggcacctggaagatgcgcccattggctggtggcctgctcaaggtggtgttcgtggtcttcgcctccttgtgtgcctggtattcggggtacctgctcgcagagctcattccagatgcacccctgtccagtgctgcctatagcatccgcagcatcggggagaggcctgtcctcaaagctccagtccccaaaaggcaaaaatgtgaccactggactccctgcccatctgacacctatgcctacaggttactcagcggaggtggcagaagcaagtacgccaaaatctgctttgaggataacctacttatgggagaacagctgggaaatgttgccagaggaataaacattgccattgtcaactatgtaactgggaatgtgacagcaacacgatgttttgatatgtatgaaggtgataactctggaccgatgacaaagtttattcagagtgctgctccaaaatccctgctcttcatggtgacctatgacgacggaagcacaagactgaataacgatgccaagaatgccatagaagcacttggaagtaaagaaatcaggaacatgaaattcaggtctagctgggtatttattgcagcaaaaggcttggaactcccttccgaaattcagagagaaaagatcaaccactctgatgctaagaacaacagatattctggctggcctgcagagatccagatagaaggctgcatacccaaagaacgaagctgacactgcagggtcctgagtaaatgtgttctgtataaacaaatgcagctggaatcgctcaagaatcttatttttctaaatccaacagcccatatttgatgagtattttgggtttgttgtaaaccaatgaacatttgctagttgtatcaaatcttggtacgcagtatttttataccagtattttatgtagtgaagatgtcaattagcaggaaactaaaatgaatggaaattcttaaagggaatgatgtgattcaagctggaaagagggttgggagaaacagcttgtccaggtggagctatgttatgatcagatcgaagtgtgacccctgtgtggtccagacagccctgcagagagaaaacctttattccattatcaccaagcacctcctagtttccgacagtcatctccttctgctgggagaattagcagcagttcagggggcttatgttatgtccttgttcaactcaacttgagctcttgaactcctcctgtgggcctgtgaatgtattcattcattccacaactctgggtg

SEQ ID NO: 6 - protein, 235 aa, Homo sapiens
MRPLAGGLLKVVFVVFASLCAWYSGYLLAELIPDAPLSSAAYSIRSIGERPVLKAPVPKRQKCDHWTPCPSDTYAYRLLSGGGRSKYAKICFEDNLLMGEQLGNVARGINIAIVNYVTGNVTATRCFDMYEGDNSGPMTKFIQSAAPKSLLFMVTYDDGSTRLNNDAKNAIEALGSKEIRNMKFRSSWVFIAAKGLELPSEIQREKINHSDAKNNRYSGWPAEIQIEGCIPKERS

(19) United States
(12) Patent Application Publication
(10) Pub. No.: US 2023/0151431 A1
(43) Pub. Date: May 18, 2023

(54) METHODS AND COMPOSITIONS FOR IDENTIFYING AND TREATING GLUTAMINASE INHIBITOR-SENSITIVE CANCERS

(71) Applicants: DANA-FARBER CANCER INSTITUTE, INC., Boston, MA (US); THE BROAD INSTITUTE, INC., Cambridge, MA (US)

(72) Inventors: Steven Corsello, Boston, MA (US); Caitlin Harrington, Cambridge, MA (US); Todd R. Golub, Cambridge, MA (US)

(73) Assignees: DANA-FARBER CANCER INSTITUTE, INC., Boston, MA (US); THE BROAD INSTITUTE, INC., Cambridge, MA (US)

(21) Appl. No.: 17/906,115

(22) PCT Filed: Mar. 12, 2021
(86) PCT No.: PCT/US2021/022099

§ 371 Date: Sep. 12, 2022

Related U.S. Application Data
(60) Provisional application No. 62/989,349, filed on Mar. 13, 2020.

(51) Int. Cl. C12Q 1/6886; G01N 33/574

(52) U.S. Cl. CPC C12Q 1/6886; G01N 33/57496; C12Q 2600/106; C12Q 2600/158; C12Q 2600/156

(57) ABSTRACT

The present disclosure relates to compositions and methods for the diagnosis and treatment or prevention of cancers that exhibit elevated expression of the glutamate/cysteine transporter SLC7A11, reduced expression of the fatty acid transporter SLC25A45 and/or reduced expression of FAM3 metabolism regulating signaling molecule B (FAM3B). In particular, the instant disclosure provides for identification of a cancer as possessing elevated SLC7A11 expression, reduced expression of SLC25A45 and/or reduced expression of FAM3B, and selecting and/or administering a glutaminase inhibitor as a therapeutic agent for such a cancer and/or subject having or at risk of developing such a cancer. Methods and compositions for therapies that combine such selection of cancers/subjects for glutaminase inhibitor therapy with other cancer therapies and/or chemotherapeutic agents are also provided.

### CROSS REFERENCE TO RELATED APPLICATION

This application claims priority to U.S. Provisional Application No. 62/989,349, filed Mar. 13, 2020, and entitled “Methods and Compositions for Identifying and Treating Glutaminase Inhibitor-Sensitive Cancers.” The entire contents of the aforementioned application are incorporated herein by reference.

### FIELD OF THE INVENTION

The invention relates generally to methods, compositions and kits for identifying and treating glutaminase inhibitor-sensitive cancers.

### SEQUENCE LISTING

The instant application contains a Sequence Listing which has been filed electronically in ASCII format and is hereby incorporated by reference in its entirety. Said ASCII copy, created on Mar. 12, 2021, is named 52199_557001WO_SeqListing_ST25.txt and is 26 kB in size.

### BACKGROUND OF THE INVENTION

Identifying therapeutic compounds capable of killing neoplastic cells that have either developed or are at risk of developing resistance to primary therapies poses an ongoing challenge for the oncology field. A need exists for agents that are capable of blocking neoplastic cells from developing chemotherapeutic resistance and/or that are capable of killing neoplastic cells that have developed resistance to primary therapies.

### BRIEF SUMMARY OF THE INVENTION

The current disclosure relates, at least in part, to the identification of cancer biomarkers capable of identifying a cancer as susceptible/responsive to one or more glutaminase inhibitors as a cancer therapy. A number of glutaminase inhibitors have been described as cancer therapeutic agents, including Telaglenastat (CB-839), which is currently in phase 2 clinical trials. Telaglenastat has also received FDA fast-track designation. When administered as a monotherapy in phase 1 clinical trials, however, only occasional responses to Telaglenastat were observed, across multiple solid tumor and hematopoietic cancer types. In view of the overall low response rate and lack of extant predictive response biomarkers, the instant disclosure has addressed a need for discovery of biomarkers possessing predictive value for identifying glutaminase inhibitor-responsive cancers. In particular, a PRISM multiplex cancer cell line screening assay has been employed herein and has discovered elevated expression of the glutamate/cysteine transporter SLC7A11 and reduced expression of the fatty acid transporter SLC25A45, as both identifying cancer cells possessing glutaminase inhibitor sensitivity. In addition, the FAM3 metabolism regulating signaling molecule B (FAM3B) was found to be indicative of cancer cell glutaminase inhibitor sensitivity. Compositions and methods for the diagnosis and treatment of subjects and/or cancers that are identified as likely to be responsive to treatment with Telaglenastat and other glutaminase inhibitors are therefore provided.

In one aspect, the instant disclosure provides a method for selecting a treatment for a subject having or at risk of developing a cancer, the method involving (a) providing or having provided a sample from a subject having or at risk of developing a cancer; (b) identifying or having identified the sample as glutaminase inhibitor-sensitive if one or more of the following is observed in the sample: (i) elevated levels of SLC7A11 mRNA or protein, as compared to an appropriate control and/or amplification of the SLC7A11 locus; (ii) reduced levels of SLC25A45 mRNA or protein, as compared to an appropriate control and/or mutation of the SLC25A45 locus, as compared to an appropriate control, (iii) reduced levels of FAM3B mRNA or protein, as compared to an appropriate control and/or mutation of the FAM3B locus, as compared to an appropriate control, thereby identifying the sample as glutaminase inhibitor-sensitive; and (c) selecting a glutaminase inhibitor for treatment of the subject so identified as providing a glutaminase inhibitor-sensitive sample, thereby selecting a treatment for said subject.

In one embodiment, the cancer is a solid tumor or a hematopoietic cancer. Optionally, the cancer is a kidney, lung, pancreas, esophageal, or gastric cancer. In certain embodiments, the cancer is an advanced and/or metastatic cancer.

In certain embodiments, identifying or having identified the sample as glutaminase inhibitor-sensitive involves identifying or having identified the presence in the sample of elevated SLC7A11 mRNA levels and reduced SLC25A45 and/or FAM3B mRNA levels, as compared to an appropriate control.

In one embodiment, the mutation of the SLC25A45 locus disrupts SLC25A45 mRNA or protein function.

In another embodiment, the mutation of the FAM3B locus disrupts FAM3B mRNA or protein function.

In some embodiments, the KGA isoform of glutaminase is inhibited. Optionally, the glutaminase inhibitor is Telaglenastat (CB-839), BPTES (Bis-2-(5-phenylacetamido-1,3,4-thiadiazol-2-yl)ethyl sulfide) or Glutaminase-IN-1, or is a pharmaceutically acceptable salt, ester, amide, prodrug or stereoisomer thereof, or a derivative thereof.

In one embodiment, the method further involves administering or having administered the selected glutaminase inhibitor to the subject.

In certain embodiments, a non-glutaminase inhibitor chemotherapeutic drug is also selected as a treatment for the subject.

In an embodiment, identifying or having identified the sample as glutaminase inhibitor-sensitive involves use of a kit as disclosed herein.

In certain embodiments, the subject is human.

Another aspect of the instant disclosure provides a method for treating or preventing cancer in a subject, the method involving: (a) providing or having provided a sample from a subject having or at risk of developing cancer; (b) identifying or having identified the sample as glutaminase inhibitor-sensitive if one or more of the following is observed in the sample: (i) elevated levels of SLC7A11 mRNA or protein, as compared to an appropriate control and/or amplification of the SLC7A11 locus; (ii) reduced levels of SLC25A45 mRNA or protein, as compared to an appropriate control and/or mutation of the SLC25A45 locus, as compared to an appropriate control, (iii) reduced levels of FAM3B mRNA or protein, as compared to an appropriate control and/or mutation of the FAM3B locus, as compared to an appropriate control, thereby identifying or having identified the sample as glutaminase inhibitor-sensitive; and (c) administering or having administered a glutaminase inhibitor to the subject.

An additional aspect of the instant disclosure provides a method for treating a subject having a cancer that is resistant to a non-glutaminase inhibitor chemotherapeutic drug, the method involving identifying one or more of the following in the cancer of the subject: (i) elevated levels of SLC7A11 mRNA or protein, as compared to an appropriate control and/or amplification of the SLC7A11 locus; (ii) reduced levels of SLC25A45 mRNA or protein, as compared to an appropriate control and/or mutation of the SLC25A45 locus, as compared to an appropriate control; (iii) reduced levels of FAM3B mRNA or protein, as compared to an appropriate control and/or mutation of the FAM3B locus, as compared to an appropriate control; and administering or having administered to the subject a glutaminase inhibitor.

One aspect of the instant disclosure provides a kit for identifying elevated expression of SLC7A11 mRNA or protein in a sample, the kit consisting essentially of an oligonucleotide for detection of SLC7A11 mRNA or an anti-SLC7A11 antibody, and instructions for its use. Optionally, the anti-SLC7A11 antibody is labeled or the kit includes a labeled secondary antibody that binds the anti-SLC7A11 antibody.

Another aspect of the instant disclosure provides a kit for identifying reduced expression of SLC25A45 or FAM3B mRNA or protein in a sample, the kit consisting essentially of an oligonucleotide for detection of SLC25A45 mRNA, an oligonucleotide for detection of FAM3B mRNA, an anti-SLC25A45 antibody, or an anti-FAM3B antibody, and instructions for its use. Optionally, the anti-SLC25A45 antibody or anti-FAM3B antibody is labeled, or the kit includes a labeled secondary antibody that binds the anti-SLC25A45 or anti-FAM3B antibody.

An additional aspect of the instant disclosure provides a kit for identifying elevated expression of SLC7A11 mRNA or protein and reduced expression of SLC25A45 mRNA or protein in a sample, the kit consisting essentially of: (1) an oligonucleotide for detection of SLC7A11 mRNA or an anti-SLC7A11 antibody; and (2) an oligonucleotide for detection of SLC25A45 mRNA or an anti-SLC25A45 antibody; and/or (3) an oligonucleotide for detection of FAM3B mRNA or an anti-FAM3B antibody, and instructions for its use.

In certain embodiments, the sample is a cancer sample.

In an embodiment, the sample is a tissue sample of a subject having a solid tumor or a hematopoietic cancer.

Another aspect of the instant disclosure provides a pharmaceutical composition for treating a subject having a cancer that exhibits one or more of the following: (1) elevated expression of SLC7A11 mRNA or protein, as compared to an appropriate control and/or amplification of the SLC7A11 locus; and/or (2) reduced levels of SLC25A45 mRNA or protein, as compared to an appropriate control and/or mutation of the SLC25A45 locus, as compared to an appropriate control; and/or (3) reduced levels of FAM3B mRNA or protein, as compared to an appropriate control and/or mutation of the FAM3B locus, as compared to an appropriate control, the pharmaceutical composition including a therapeutically effective amount of a glutaminase inhibitor and a pharmaceutically acceptable carrier.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof or cellular therapeutics such as allogeneic transplantation and/or CART-cell therapy.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, solid tumor and hematopoietic cancers, including, e.g., lung cancer (e.g., bronchogenic carcinoma, small cell lung cancer (SCLC), non-small cell lung cancer (NSCLC), adenocarcinoma of the lung); brain cancer (e.g., meningioma, glioblastomas, glioma (e.g., astrocytoma, oligodendroglioma), medulloblastoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); Ewing's sarcoma; ocular cancer (e.g., intraocular melanoma, retinoblastoma); familiar hypereosinophilia; gall bladder cancer; gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); myeloid malignancies (e.g., acute myeloid leukemia (AML) (e.g., B-cell AML, T-cell AML), myelodysplastic syndrome, myeloproliferative neoplasm, chronic myelomonocytic leukemia (CMML) and chronic myelogenous leukemia (CML) (e.g., B-cell CML, T-cell CML)) and lymphocytic leukemia such as acute lymphocytic leukemia (ALL) (e.g., B-cell ALL, T-cell ALL) and chronic lymphocytic leukemia (CLL) (e.g., B-cell CLL, T-cell CLL); melanoma and ovarian cancer (e.g., cystadenocarcinoma, ovarian embryonal carcinoma, ovarian adenocarcinoma, clear cell ovarian cancer), colorectal cancer (e.g., colon cancer, rectal cancer, colorectal adenocarcinoma), endometrial cancer (e.g., uterine cancer, uterine sarcoma), esophageal cancer (e.g., adenocarcinoma of the esophagus, Barrett's adenocarcinoma), and gastric cancer (e.g., stomach adenocarcinoma (STAD)), including, e.g., colon adenocarcinoma (COAD), oesophageal carcinoma (ESCA), rectal adenocarcinoma (READ) and uterine corpus endometrial carcinoma (UCEC). Other exemplary forms of cancer include, but are not limited to, diffuse large B-cell lymphoma (DLBCL), as well as the broader class of lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); and a mixture of one or more leukemia/lymphoma as described above.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The terms “elevated expression,” “over-expression,” or “elevated levels”, as used herein with respect to mRNA and/or protein levels, refer to an elevated level of the mRNA and/or protein being assessed, as compared to an appropriate control (e.g., control sample and/or control value). In certain embodiments, the magnitude of the elevated level of mRNA and/or protein with respect to an appropriate control can be, e.g., at least 1.2×, at least 1.3×, at least 1.4×, at least 1.5×, at least 1.6×, at least 1.7×, at least 1.8×, at least 1.9×, at least 2×, at least 3×, at least 4×, at least 5×, at least 6×, at least 7×, at least 8×, at least 9×, at least 10×, at least 50×, at least 100×, at least 10³×, at least 10⁴×, or more.

The terms “reduced expression,” “under-expression,” or “reduced levels”, as used herein with respect to mRNA and/or protein levels, refer to a reduced level of the mRNA and/or protein with respect to an appropriate control (e.g., control sample and/or control value). In certain embodiments, the magnitude of the reduced level of mRNA and/or protein with respect to an appropriate control can be, e.g., at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, at least 99.5%, or can be a complete reduction (below the threshold of detection employed).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, er al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

As used herein, the term “non-glutaminase inhibitor chemotherapeutic drug” refers to any drug that can be employed in cancer therapy that is not an inhibitor of glutaminase.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.).

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows the predictions obtained herein via PRISM screening for Telaglenastat biomarkers. In 500 adherent cell lines, multivariate models concluded that the top two important features in predicting Telaglenastat tumor cell line sensitivity were high expression of SLC7A11 and low expression of SLC25A45. In the plot of FIG. 1, green depicts lower expression associated with killing and red depicts higher expression associated with killing. For biomarker analysis, multiple ATLANTIS predictive models were trained for the Telaglenastat PRISM profile. Cross-validated R square values and Pearson scores were then reported and feature importance values were determined.

FIG. 2 shows that high SLC7A11 expression and low SLC25A45 expression correlated with Telaglenastat tumor cell killing in PRISM. Using Pearson correlations and associated p-values, univariate analysis between Telaglenastat sensitivity and genomic features revealed significant associations between SLC7A11 (top panel) and SLC25A45 (middle panel) expression levels and Telaglenastat-induced cell-line killing. The Benjamin Hochberg algorithm was used to compute q values from p values. Compound sensitivity is defined as log₂ fold change in viability compared to DMSO treatment. Volcano plot data (bottom panel) depict AUC values from MTS011 500 adherent cell lines.

FIG. 3 shows that glutaminase (GLS) knockout was the top-correlated CRISPR feature in the Telaglenastat PRISM profile, which indicated Telaglenastat on-target activity. The volcano plot (top panel) shows the positive association between Telaglenastat AUC values and CRISPR knockout scores. At bottom, Telaglenastat-sensitive cell line survival was demonstrated to be highly dependent on GLS.

FIG. 4 shows a schematic of the pathways by which, without wishing to be bound by theory, SLC7A11 elevation and SLC25A45 reduction may confer sensitivity to glutaminase inhibitors and subsequent starvation of cancer cells. Shown is CB839, an exemplary glutaminase inhibitor, whose effect on glutamate levels and subsequent ATP production is enhanced by increased SLC7A11, likely through SLC7A11 mediated increase in cysteine to glutamate exchange, which reduces the amount of alpha-ketoglutarate entering the Krebs/tricarboxylic acid cycle (TCA). Also indicated are the effects of SLC25A45 activity, which transports acyl carnitine, in combination with glutaminase inhibitors. Thus, both high expression of SLC7A11 and low expression of SLC25A45 result in a lower level of metabolites entering the TCA, thereby lowering available ATP, which is believed to be the mechanism by which glutaminase-inhibitor cell death is potentiated.

FIG. 5 shows the high sensitivity of LU99 cells to Telaglenastat. The percent viability of LU99 cells is plotted vs. the log concentration of Telaglenastat and Paclitaxel. The IC50 of Telaglenastat (1.2 nM) was on the order of that of Paclitaxel, a potent anti-mitotic drug (2.2 nM).

FIG. 6 shows that SLC25A45 overexpression and SLC7A11 knock-out rescued LU99 cell viability during Telaglenastat treatment. FIG. 6, top, is a graph that shows cell viability in response to Telaglenastat concentration, in LU99 wild type cells and in LU99 cells over-expressing SCL25A45 via a SCL25A45 304 construct. FIG. 6, middle, is a graph that shows a comparison of cell viability in response to Telaglenastat concentration, in LU99 cells over-expressing SCL25A45, in SLC7A11 knock-out LU99 cells (more detail is shown in FIGS. 7A and 7B, below), and in LU99 wild type cells. FIG. 6, bottom, shows a table of Telaglenastat IC50 values observed in SLC7A11g2 knock-out LU99 cells and in SLC25A45 over-expressing LU99 cells.

FIGS. 7A and 7B demonstrate that knock-out of SLC7A11 in LU99 cells rescued such cells from Telaglenastat-induced cell killing. Lu99 cells exhibited sensitivity to multiple GLS inhibitors. However, sensitivity to 968 was not rescued by knock-out of SLC7A11, which indicated that inhibition of the KGA isoform (NCBI identifiers: NP_055720.3, NM_014905.4, uniprot identifier: 094925-1) rather than the GAC isoform (NCBI identifiers: NP_001243239.1, NM_001256310.1, uniprot identifier: 094925-3) of glutaminase was necessary for 968-induced cell killing. Shown are plots of Lu99 cells with knock-outs of glutaminase, SLC7A11, or LacZ (control) vs glutaminase inhibitor log concentration. Top panel: Telaglenastat, second panel: 968; third panel: BPTES (Bis-2-(5-phenylacetamido-1,3,4-thiadiazol-2-yl)ethyl sulfide); and bottom panel: Glutaminase IN 1. FIG. 7B shows a western blot which confirmed the knock-out efficiency of SLC7A11 in LU99 cells, as well as a table showing the effect of SLC7A11 knock-out vs LacZ (control) knock-out upon the observed IC50 values for indicated glutaminase inhibitors in LU99 cells.

FIGS. 8A and 8B show that addition of alpha ketoglutarate to LU99 cell media also rescued Telaglenastat-induced cell death. Shown are the percent viability values observed for LU99 cells treated with glutaminase inhibitor (response vs the log concentration (M) of glutaminase inhibitor is shown), in the presence and absence of 2 mM alpha ketoglutarate. FIG. 8A shows plotted results for Telaglenastat; 968; BPTES; Glutaminase IN 1; and Paclitaxel (as a non-glutaminase inhibitor control). FIG. 8B displays tabulated IC50 values for the results of FIG. 8A, which show that, apart from 968, the IC50 of each glutaminase inhibitor increased by many orders of magnitude with alpha ketoglutarate in the media. These data demonstrate that glutaminase inhibitors induce cell death via starvation.

FIGS. 9A and 9B show that SLC25A45 overexpression and SLC7A11 knock-out increased NCI-H2122 cell viability during Telaglenastat treatment. FIG. 9A, top, is a graph showing cell viability in response to Telaglenastat treatment at varying concentrations, in NCI-H2122 cells over-expressing SCL25A45 (via a SCL25A45 304 construct), in NCI-H2122 wild type cells, in SLC7A11 knock-out NCI-H2122 cells, and in SLC7A11 knock-out NCI-H2122 cells also over-expressing SCL25A45. Notably, with both SLC7A11 knock-out and SCL25A45 over-expression, the NCI-H122 cells were less sensitive to Telaglenastat, with the combination of SLC7A11 knock-out and SCL25A45 over-expression rendering the NCI-H122 cells almost completely resistant to Telaglenastat. FIG. 9A, second from top, is a graph comparing cell viability in response to Glutaminase IN 1 treatment, in NCI-H2122 wild type cells and in SLC7A11 knock-out NCI-H2122 cells. FIG. 9A, third from top, is a graph comparing cell viability in response to treatment with the glutaminase inhibitor 968, in NCI-H2122 wild type cells and in SLC7A11 knock-out NCI-H2122 cells. The penultimate graph of FIG. 9A compares cell viability in response to BPTES treatment, in NCI-H2122 wild type cells and in SLC7A11 knock-out NCI-H2122 cells. The final graph of FIG. 9A compares cell viability in response to Paclitaxel treatment, in NCI-H2122 wild type cells and in SLC7A11 knock-out NCI-H2122 cells. FIG. 9A, bottom, is a table showing the level of SLC25A45 mRNA expression in NCIH122 cells over-expressing SCL25A45, and in the SLC7A11 knock-out NCIH122 cells also over-expressing SLC25A45. FIG. 9B, top, is a table showing the IC50 of Telaglenastat, the 968 glutaminase inhibitor, BPTES, Glutaminase IN 1, and Paclitaxel in NCIH122 cells (SLC71A11g2 knock-out cells, SLC25A45 over-expressing cells, SLC71A11g2 knock-out also over-expressing SLC25A45 and wild type cells). FIG. 9B, bottom, is a western blot that confirms the presence of SLC7A11 in NCI-H122 wild type cells (lane 3), and the absence of SLC7A11 in SLC7A11 knock-out cells (lanes 1 and 2). The SLC7A11 knockout was thereby confirmed, while SLC25A45 overexpression was confirmed by qPCR (data not shown).

FIG. 10 shows a volcano plot that depicts the results of a CRISPR knockout screen performed on LU99 cells under Telaglenastat treatment. The genome-wide CRISPR knockout screen was performed on cells treated with Telagenastat for 9 days, with cell viability (cell death) observed. The significance of the knock-out result is plotted on the y axis against the log-fold change (LFC) of the sgRNA-mediated knock-out effect on the x-axis. Notably, the observed SLC7A11 knock-out response to Telaglenastat was both highly significant and exhibited a high LFC in cell death.

FIG. 11 shows that ectopic expression of FAM3B also increased LU99 cell viability under Telaglenastat treatment. FIG. 11, at top, shows a graph that depicts cell viability in response to Telaglenastast treatment in LU99 wild type cells and in LU99 cells with ectopic expression of FAM3B. FIG. 11, second from top, is a graph that depicts cell viability in response to Paciltaxel treatment in LU99 wild type cells and in LU99 cells with ectopic expression of FAM3B. FIG. 11, at bottom left, shows a western blot that specifically identified actin (top) and a V5-tag (bottom) present on FAM3B in a V5-FAM3B construct, assayed in LU99 cells harboring a V5-FAM3B construct, LU99 wild type cells, and a TE6 cell line that expressed the FAM3B ORF without the V5 tag. FIG. 11, at bottom right, shows a table that presents the observed IC50 values for Telaglenastat treatment in LU99 wild type cells and in LU99 cells with ectopic expression of FAM3B.

### DETAILED DESCRIPTION OF THE INVENTION

The present disclosure is directed, at least in part, to the discovery herein of biomarkers capable of identifying cancer cells that are sensitive to glutaminase inhibitor-mediated cell killing. Glutaminase inhibitors have been successful in sporadically treating multiple solid tumor and hematopoietic cancers. Phase 1 clinical trial observations have led to one glutaminase inhibitor, Telaglenastat, reaching phase 2 trials as a combination therapy. However, because of the sporadic nature of Telaglenastat efficacy in clinical trials performed to date, methods for identifying cancer cells and tissue(s) that possess glutaminase inhibitor sensitivity, as are disclosed herein, can significantly improve the accuracy of the therapeutic application of glutaminase inhibitors in cancer treatment. The instant disclosure specifically describes the instant discovery of SLC7A11, SLC25A45 and FAM3B as predictive biomarkers, a discovery achieved using PRISM multiplex cancer cell line viability assays.

PRISM is a high throughput screening assay through which 5,000 small molecules were tested against 500 cancer cell lines. Using this assay, Telaglenastat (CB-839) was found to selectively kill cell lines across a broad range of lineages. Without wishing to be bound by theory, Telaglenastat works by inhibiting Glutaminase (GLS), a critical amidohydrolase enzyme responsible for converting glutamine to glutamate in the cell. Cells rely on glutamate to support growth, metabolism, and proliferation. Selective Telaglenastat activity was associated with baseline solute carrier expression. In particular, SLC7A11, a specific glutamate/cysteine transporter, was expressed at high levels in sensitive cell lines. SLC25A45, a fatty acid transporter in the mitochondria, was expressed at low levels. Utilizing CRISPR/Cas9 knockout cell lines and overexpressing open reading frames, functional roles for SLC7A11, SLC25A45 and FAM3B were validated in glutaminase inhibitor-mediated killing. Biomarker validation in vivo enables identification of patients who could benefit from treatment.

The instant disclosure provides compositions and methods for the diagnosis and treatment of cancer that employ glutaminase inhibitors or derivatives thereof, either alone (i.e., as a monotherapy, optionally in certain classes of cancer) or in combination with other chemotherapeutic drugs.

The instant discovery was made using large-scale multiplex profiling of the glutaminase inhibitor Telaglenastat against 500 cancer cell lines, employing a PRISM multiplexed cellular viability assay. It was found that high expression of SLC7A11 and low expression of SLC25A45 carrier proteins correlated with Telaglenastat sensitivity. In confirmatory studies, it was identified that 1) glutaminase knockout was the most correlated CRISPR effect with Telaglenastat sensitivity 2) over-expression of SLC25A45 rescued Telaglenastat sensitivity 3) knockout of SLC7A11 rescued sensitivity to glutaminase inhibitors Telaglenastat, BPTES, and Glutaminase IN 1, and 4) alpha ketoglutarate added to cell media also rescued sensitivity to glutaminase inhibitors Telaglenastat, BPTES, and Glutaminase IN 1. In addition, over-expression of FAM3B rescued Telaglenastat sensitivity. Without wishing to be bound by theory, these results indicated that glutaminase inhibitors act on-target to starve and kill cancer cells in a manner dependent on SLC7A11, SLC25A45, and FAM3B activity.

Disclosed herein is the concept of using glutaminase inhibitors preferentially to treat cancers that exhibit high mRNA expression, high protein expression and/or amplification of SLC7A11 and low mRNA expression, low protein expression and/or mutation of SLC25A45 or FAM3B. Glutaminase inhibitor-targeted cancers include cancers that exhibit high SLC7A11 and/or low SLC25A45 or FAM3B expression at baseline or following prior cancer treatment. Target cancer types include both solid tumors and hematopoietic cancers—such cancers include, but are not limited to, e.g., colorectal cancer, kidney cancer, liver cancer and lymphoma, among others.

Exemplary glutaminase inhibitors are shown in Table 1.

TABLE 1

| | | Clinical |
|---|---|---|
| Drug Name | Chemical Structure | Development |
| Telaglenastat (CB-839) | 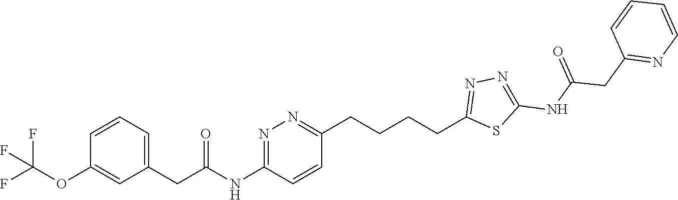 [SMILES: O=C(Cc1cccc(OC(F)(F)F)c1)Nc1ccc(CCCCc2nnc(NC(=O)Cc3ccccn3)s2)nn1] | Phase 2 |
| BPTES | 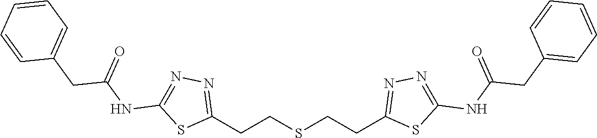 [SMILES: O=C(Cc1ccccc1)Nc1nnc(CCSCCc2nnc(NC(=O)Cc3ccccc3)s2)s1] | Pre-clinical |
| 968 | 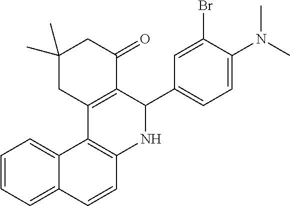 [SMILES: CN(C)c1ccc(C2Nc3ccc4ccccc4c3C3=C2C(=O)CC(C)(C)C3)cc1Br] | Pre-clinical |
| Glutaminase-IN-1 | 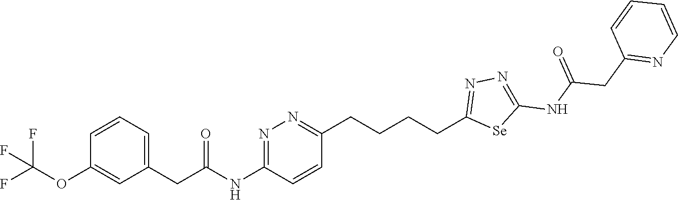 [SMILES: O=C(Cc1cccc(OC(F)(F)F)c1)Nc1ccc(CCCCc2nnc(NC(=O)Cc3ccccn3)[se]2)nn1] | Pre-clinical |

Other glutaminase inhibitors known in the art include but are not limited to:

| Drug Name | Chemical Structure |
|---|---|
| 6-Diazo-5-oxo-L- norleucine (DON) | 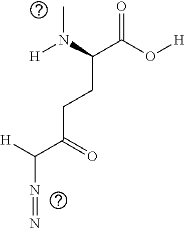 |
| Hexylselen (CPD- 3B) | 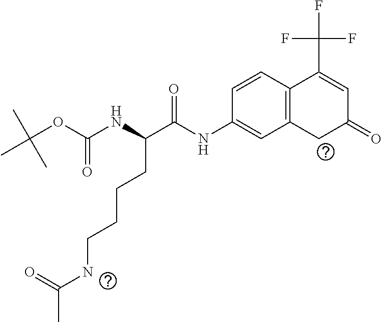 [SMILES: CC(=O)NCCCC[C@@H](NC(=O)OC(C)(C)C)C(=O)Nc1ccc2c(c1)CC(=O)C=C2C(F)(F)F] |
| Physapubescin | 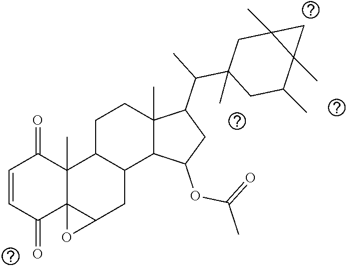 [SMILES: CC(=O)OC1CC(C(C)C2(C)CC(C)C3(C)CC3(C)C2)C2(C)CCC3C(CC4OC45C(=O)C=CC(=O)C35C)C12] |

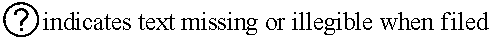

Glutaminase inhibitors known in the art include those described, e.g., in WO 2015/192014; U.S. Pat. No. 9,783,533; U.S. Patent Publication No. 2016/0297761; U.S. Pat. No. 10,441,587; and U.S. Patent Publication No. 2016/0010158, among others.

In certain embodiments, a glutaminase inhibitor of the instant disclosure comprises a compound of formula (I):

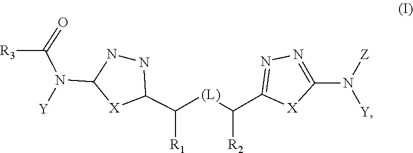

or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or

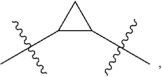 [SMILES: *C1CC1*]

preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂. CH₂CH₂CH₂ or CH₂ may be replaced by hydroxy;
X, independently for each occurrence, represents S, O or CH═CH, preferably S or CH═CH, wherein any hydrogen atom of a CH unit may be replaced by alkyl:
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy;
R₃, independently for each occurrence, represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₅, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H.
Identification of SLC7A11 Overexpressing and/or SLC7A11 Amplified Cells. Tissues and/or Cancers, and/or SLC25A45 or FAM3B Underexpressing and/or SLC25A45 or FAM3B Depleted Cells, Tissues and/or Cancers

Identification of a tissue, tumor and/or cancer of a subject as exhibiting amplification of the SLC7A11 locus and/or elevated levels of SLC7A11 expression (including SLC7A11 overexpression), and/or identification of a tissue, tumor and/or cancer of a subject as exhibiting mutation of the SLC25A45 or FAM3B locus and/or reduced levels of SLC25A45 or FAM3B expression (including SLC25A45 or FAM3B under-expression), can be performed by any method available in the art. Gene/genomic amplification events can be identified via genomic sequencing and/or genotyping approaches (including next-generation sequencing approaches), among others. Certain methods and compositions described herein relate to identification of a cell, cell line, sample, tissue and/or subject having or at risk of developing a cancer that is likely to be responsive to administration of a glutaminase inhibitor as exhibiting elevated levels of SLC7A11 expression (including SLC7A11 overexpression) at the mRNA or protein level, based upon gene-specific assessment of SLC7A11 mRNA or protein performed upon the cell, cell line, sample, tissue and/or subject having or at risk of developing a cancer that exhibits elevated levels of SLC7A11 expression. Similarly, certain methods and compositions described herein relate to identification of a cell, cell line, sample, tissue and/or subject having or at risk of developing a cancer that is likely to be responsive to administration of a glutaminase inhibitor as exhibiting reduced levels of SLC25A45 or FAM3B expression (including SLC25A45 or FAM3B under-expression) at the mRNA or protein level, based upon gene-specific assessment of SLC25A45 or FAM3B mRNA or protein performed upon the cell, cell line, sample, tissue and/or subject having or at risk of developing a cancer that exhibits reduced levels of SLC25A45 or FAM3B expression. In certain embodiments, detection of elevated SLC7A11 levels or reduced SLC25A45 or FAM3B levels can readily be performed, e.g., via assessment of mRNA expression levels (e.g., via real-time PCR or other such quantitative method). In related embodiments, assessment of SLC7A11 or SLC25A45 or FAM3B mRNA expression can be performed via art-recognized, oligonucleotide-mediated approaches, including, e.g., Northern blotting, expression profiling using RT-PCR and/or next-generation sequencing performed upon cellular transcriptomes.

In some embodiments, detection of SLC25A45, SLC7A11, and/or FAM3B levels can readily be performed, e.g., via immunoassay for detection of SLC25A45, SLC7A11 or FAM3B protein levels.

Protein levels of SLC7A11, SLC25A45, and FAM3B can be quantitated in a variety of ways well known in the art, such as immunoprecipitation, Western blot analysis (immunoblotting), ELISA or fluorescence-activated cell sorting (FACS). Antibodies directed to SLC7A11, SLC25A45, and/or FAM3B can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of glutaminase inhibitor. In certain embodiments, glutaminase inhibitors may be administered in combination with an additional therapeutic agent, optionally a chemotherapeutic agent.

Exemplary human SLC25A45 mRNA and protein sequences are:

Homo sapiens solute carrier family 25 member 45
(SLC25A45), transcript variant 1, mRNA,
Accession No. NM_182556.3 (SEQ ID NO: 1)
CACAGATCCTTGAAAGGGTTTTGGTTTTGACTGAGCAGAATGGTGCCATT
GCAGATCAACACGCACTTTAAATAATCAAAACCCCTATCTTTCCCCTGGG
AAGGAGGAAGCCGAGGAAGGCTGTGTTTCTGACTCACACGGGGGAGTCGG
GGGAGTCGTAAACAACCCTGAAGAGAACAGCCAGGCCTGTTCCCGCAAGC
CCAGACAATGCCGGTGGAGGAATTTGTGGCTGGCTGGATCTCTGGCGCTC
TGGGCTTGGTCCTGGGACACCCGTTTGACACTGTAAAGGTGAGGCTGCAG
ACCCAGACCACCTACCGGGGCATCGTTGATTGCATGGTCAAGATTTACCG
CCATGAGTCCCTCCTGGGCTTCTTCAAGGGAATGAGCTTCCCCATTGCCA
GCATAGCTGTGGTCAACTCTGTCCTGTTTGGGGTCTATAGCAACACCCTG
CTGGTGCTCACGGCCACCTCCCACCAGGAGCGGCGGGCCCAGCCGCCCAG
CTACATGCACATCTTCCTAGCGGGCTGCACCGGGGGGTTCCTGCAGGCCT
ACTGTCTGGCTCCTTTTGACCTCATCAAAGTCCGGCTACAAAACCAGACA
GAGCCAAGGGCCCAGCCAGGGAGCCCCCCACCCCGGTACCAGGGGCCCGT
GCACTGTGCAGCCTCCATCTTCCGGGAGGAGGGGCCCCGGGGGCTGTTCC
GAGGAGCCTGGGCCCTGACGCTGAGGGACACCCCCACGGTGGGGATCTAC
TTCATCACCTATGAAGGGCTCTGTCGCCAGTACACACCAGAAGGCCAGAA
TCCCAGCTCAGCCACGGTGCTGGTGGCAGGGGGCTTTGCAGGCATTGCTT
CCTGGGTGGCAGCCACGCCCTTAGACGTGATCAAGTCCCGGATGCAGATG
GATGGACTGAGACGCAGAGTGTACCAGGGGATGCTGGACTGCATGGTGAG
CAGCATCCGGCAGGAAGGACTGGGAGTCTTCTTCCGGGGGGTCACCATCA
ACAGTGCCCGCGCCTTTCCCGTCAATGCTGTCACCTTCCTCAGCTACGAA
TATCTCCTCCGCTGGTGGGGATGAGCCCTGCGGCAATGCCAGCAGCTCCC
CATCAGGCCCACGGCCTGGAGGCCAGTTTGAGATTGGAGGCCAGGTTGAA
AGCTTGCAAATCAGTGCAAGAGGCTCAGCCCTTCCTAACCAAGGTGCCTC
CCACCCGCGCAGATCTGGGCTGGGCAGACACCTGTGGGAGCCGGAAGCCA
GGGGGCCTGTGCAGCCTCCCTGTGTAGCTGGCCTTGACTCCTTTGCCTCC
CACATCTGTGAAACAGGGAGCATGAGGCACAAGTGAGCTGGCAAGTGGTG
CTGGTGACATCCCAGCTCCTGTCCTGTGCCTTCACCTCTTTTTTTTTTTT
TTTTTTTTTTTGAGATGGAGTCTTTCTCTGTCACCCAGGCTGGAATGCAG
TGGCGTGATCTCGGCTCACTGCAACCTCCGCCTTTCCACCTTCCGGGTTC
AAGCAACTCTCCTGCCTCAGCCTCCCAAGTAGCTGGGACTATAGGCGCCC
GCCACCACACCCAGCTAAATTTTTGTATTTTTAGTAGAGACAGGGTTTCA
CCATGTCAGCCAGGATGGTCTTGATCTCCTGACTTTGTGATCCGCCCGCG
TCGGCCTCTCAAAGTGCTGGGATTACAGGCGTGAGCCACCGCGCCCGGCT
GCCTTCACCTCTTAAGGAGCTCTGAGACTCCACTTCTGAGAGTCCCTGCG
GCCTCCCACCTCCCTGCCTTTCAAAGCTCTCTCCCCCATGACCCAGGATA
ACCCTATGTCTCCTCCCCCAGAATCCTTCAGTGGCTCTCATCACCTTCAG
GAAAAGCCCAAACTCCTTCCACCCTCCAGGTTCCTCCCTCCCCACGAGGC
TTTGTTCTCCTGGGGTTGCTTCCTGGACCCTGAACAAGTTGTGCTCTCAT
TGGCCGGGCCTGGCCAGCAGTGCACAGTGCCTGGCAGGTTGACTCTACCA
TCCCCGGGGCTGGCCCCGCTCTCCTCCGAGACCCAGGCTGAGCCCAGTCC
CCTCACCCTTCCTTGACTTACCTCCCCACCTGAGGCTGACTTTGGGGTTC
CCAGACACCCTACCCACACACATGCCTTGATCATAGCACTTGCCTGCGCT
TCTTCAGAGTCATTAATTTGCTTCTCGGCTTCCCCACTGGACTGTGAGCT
GCCTGAGGTCAGGGATTGCGCTTTGGATGGTTTCCAGCCTGAGCCTGGTG
CTTGAACAGACGTGTGCAATAAATGCTCGTTAAATGATGAAAAAAAAAAA
AAAAAAA
Homo sapiens solute carrier family 25 member 45
(SLC25A45), isoform a protein, Accession No.
NP_872362.3 (SEQ ID NO: 2):
MPVEEFVAGWISGALGLVLGHPFDTVKVRLQTQTTYRGIVDCMVKIYRHE
SLLGFFKGMSFPIASIAVVNSVLFGVYSNTLLVLTATSHQERRAQPPSYM
HIFLAGCTGGFLQAYCLAPFDLIKVRLQNQTEPRAQPGSPPPRYQGPVHC
AASIFREEGPRGLFRGAWALTLRDTPTVGIYFITYEGLCRQYTPEGQNPS
SATVLVAGGFAGIASWVAATPLDVIKSRMQMDGLRRRVYQGMLDCMVSSI
RQEGLGVFFRGVTINSARAFPVNAVTFLSYEYLLRWWG

Exemplary human SLC7A11 mRNA and protein sequences are:

Homo sapiens solute carrier family 7 member 11 (SLC7A11), mRNA Accession No.
NM_014331.4 (SEQ ID NO: 3):
GGTTTGTAATGATAGGGCGGCAGCAGCAGCAGCAGCAGCAGTGGTGGAACGAGG
AGGTGGAGAATTGAGAGCACGATGCATACACAGGTGTTTCTGAGTAGTAATTAGA
TCGCTGTGAAGGAAAAAGCACACCTTTGAGTTTTCACCTGTGAACACTATAGCGCT
GAGAGAGACAGTCTGAAAGCAGAGGAAGACATCGATCAGTAACACCAAGAGACA
CCAAAGTTGAAAGTTTTGTTTTCTTTCCCTCTGTTTTATTTTTCCCCCGTGTGTCCCT
ACTATGGTCAGAAAGCCTGTTGTGTCCACCATCTCCAAAGGAGGTTACCTGCAGGG
AAATGTTAACGGGAGGCTGCCTTCCCTGGGCAACAAGGAGCCACCTGGGCAGGAG
AAAGTGCAGCTGAAGAGGAAAGTCACTTTACTGAGGGGAGTCTCCATTATCATTG
GCACCATCATTGGAGCAGGAATCTTCATCTCTCCTAAGGGCGTGCTCCAGAACACG
GGCAGCGTGGGCATGTCTCTGACCATCTGGACGGTGTGTGGGGTCCTGTCACTATT
TGGAGCTTTGTCTTATGCTGAATTGGGAACAACTATAAAGAAATCTGGAGGTCATT
ACACATATATTTTGGAAGTCTTTGGTCCATTACCAGCTTTTGTACGAGTCTGGGTGG
AACTCCTCATAATACGCCCTGCAGCTACTGCTGTGATATCCCTGGCATTTGGACGC
TACATTCTGGAACCATTTTTTATTCAATGTGAAATCCCTGAACTTGCGATCAAGCTC
ATTACAGCTGTGGGCATAACTGTAGTGATGGTCCTAAATAGCATGAGTGTCAGCTG
GAGCGCCCGGATCCAGATTTTCTTAACCTTTTGCAAGCTCACAGCAATTCTGATAA
TTATAGTCCCTGGAGTTATGCAGCTAATTAAAGGTCAAACGCAGAACTTTAAAGAC
GCCTTTTCAGGAAGAGATTCAAGTATTACGCGGTTGCCACTGGCTTTTTATTATGG
AATGTATGCATATGCTGGCTGGTTTTACCTCAACTTTGTTACTGAAGAAGTAGAAA
ACCCTGAAAAAACCATTCCCCTTGCAATATGTATATCCATGGCCATTGTCACCATT
GGCTATGTGCTGACAAATGTGGCCTACTTTACGACCATTAATGCTGAGGAGCTGCT
GCTTTCAAATGCAGTGGCAGTGACCTTTTCTGAGCGGCTACTGGGAAATTTCTCAT
TAGCAGTTCCGATCTTTGTTGCCCTCTCCTGCTTTGGCTCCATGAACGGTGGTGTGT
TTGCTGTCTCCAGGTTATTCTATGTTGCGTCTCGAGAGGGTCACCTTCCAGAAATCC
TCTCCATGATTCATGTCCGCAAGCACACTCCTCTACCAGCTGTTATTGTTTTGCACC
CTTTGACAATGATAATGCTCTTCTCTGGAGACCTCGACAGTCTTTTGAATTTCCTCA
GTTTTGCCAGGTGGCTTTTTATTGGGCTGGCAGTTGCTGGGCTGATTTATCTTCGAT
ACAAATGCCCAGATATGCATCGTCCTTTCAAGGTGCCACTGTTCATCCCAGCTTTG
TTTTCCTTCACATGCCTCTTCATGGTTGCCCTTTCCCTCTATTCGGACCCATTTAGTA
CAGGGATTGGCTTCGTCATCACTCTGACTGGAGTCCCTGCGTATTATCTCTTTATTA
TATGGGACAAGAAACCCAGGTGGTTTAGAATAATGTCGGAGAAAATAACCAGAAC
ATTACAAATAATACTGGAAGTTGTACCAGAAGAAGATAAGTTATGAACTAATGGA
CTTGAGATCTTGGCAATCTGCCCAAGGGGAGACACAAAATAGGGATTTTTACTTCA
TTTTCTGAAAGTCTAGAGAATTACAACTTTGGTGATAAACAAAAGGAGTCAGTTAT
TTTTATTCATATATTTTAGCATATTCGAACTAATTTCTAAGAAATTTAGTTATAACT
CTATGTAGTTATAGAAAGTGAATATGCAGTTATTCTATGAGTCGCACAATTCTTGA
GTCTCTGATACCTACCTATTGGGGTTAGGAGAAAAGACTAGACAATTACTATGTGG
TCATTCTCTACAACATATGTTAGCACGGCAAAGAACCTTCAAATTGAAGACTGAGA
TTTTTCTGTATATATGGGTTTTGTAAAGATGGTTTTACACACTATAGATGTCTATAC
TGTGAAAAGTGTTTTCAATTCTGAAAAAAAGCATACATCATGATTATGGCAAAGA
GGAGAGAAAGAAATTTATTTTACATTGACATTGCATTGCTTCCCCTTAGATACCAA
TTTAGATAACAAACACTCATGCTTTAATGGATTATACCCAGAGCACTTTGAACAAA
GGTCAGTGGGGATTGTTGAATACATTAAAGAAGAGTTTCTAGGGGCTACTGTTTAT
GAGACACATCCAGGAGTTATGTTTAAGTAAAAATCCTTGAGAATTTATTATGTCAG
ATGTTTTTTCATTCATTATCAGGAAGTTTTAGTTATCTGTCATTTTTTTTTTTCACAT
CAGTTTGATCAGGAAAGTGTATAACACATCTTAGAGCAAGAGTTAGTTTGGTATTA
AATCCTCATTAGAACAACCACCTGTTTCACTAATAACTTACCCCTGATGAGTCTAT
CTAAACATATGCATTTTAAGCCTTCAAATTACATTATCAACATGAGAGAAATCACC
AACAAAGAAGATGTTCAAAATAATAGTCCCATATCTGTAATCATATCTACATGCAA
TGTTAGTAATTCTGAAGTTTTTTAAATTTATGGCTATTTTTACACGATGATGAATTT
TGACAGTTTGTGCATTTTCTTTATACATTTTATATTCTTCTGTTAAAATATCTCTTCA
GATGAAACTGTCCAGATTAATTAGGAAAAGGCATATATTAACATAAAAATTGCAA
AAGAAATGTCGCTGTAAATAAGATTTACAACTGATGTTTCTAGAAAATTTCCACTT
CTATATCTAGGCTTTGTCAGTAATTTCCACACCTTAATTATCATTCAACTTGCAAAA
GAGACAACTGATAAGAAGAAAATTGAAATGAGAATCTGTGGATAAGTGTTTGTGT
TCAGAAGATGTTGTTTTGCCAGTATTAGAAAATACTGTGAGCCGGGCATGGTGGCT
TACATCTGTAATCCCAGCACTTTGGGAGGCTGAGGGGGTGGATCACCTGAGGTCG
GGAGTTCTAGACCAGCCTGACCAACATGGAGAAACCCCATCTCTACTAAAAATAC
AAAATTAGCTGGGCATGGTGGCACATGCTGGTAATCTCAGCTATTGAGGAGGCTG
AGGCAGGAGAATTGCTTGAACCCGGGAGGCGGAGGTTGCAGTGAGCCAAGATTGC
ACCACTGTACTCCAGCCTGGGTGACAAAGTCAGACTCCATCTCCAAAAAAAAAAG
ATTATATATATATATATATGTGTGTGTATGTGTGTGTGTGTGTGTGTGTGTATATAT
ATATATATATATATACACACACACACACACACTTTTTATATATATATATATATATAT
ATAGTGGAACTTACAAATGAGAGTAATATAATGATGAAATTTTGAACTGTTATTTA
TAAACATCTAAGGTAAAATGGTTAGTCATGGCCAGAGTATGTTTCATCCTTTAATT
TTTGTCCATTTGAAAATAAGGATTTTTGAAAGAATTATACCAATTAAAATTATTAA
AGGCAAACATAGAATTCATAAAAAATTGTCCAAAGTAGAAATGATGACCTATAAT
TTGGAGCATTTCCAATTCAGTAATTTCAATTTTGCTCTTGAAAACATTTAATATATA
TCCAAGACTGACATTTCTTTAGCTGAACCTAACGTTTGGGTCTCTGAGTGAATTTAT
AATAACTCCTTCCTTCCTTAGCATAGGGTTTTCAAAATTTGATTTATAATTCCTATT
TCCAGTAAATATTGTTCATTTGTCCACATCTCTCCCTATGATATGTTGCTGGAGGTA
AGAATTTCTTTCATATTCCTATTTTTTTTTTCCCCATAGACTAGGCTCATAGAATTTA
AACAAGCAAATTTTCCTGAGCTTTTTCTTGCCAAATGAAAGAAGACTGGTAAATTC
TCATAGAGAGGTTTGTGTAGTTCTTGGCTCTTCCTGGGGTTAATGTGCTTATATTCA
CAGTGGCAAATTGGTCTCAGACTTTAATTTATTTATTTTTGATTTGAATTTCTCTTTA
AAAGTATCAATTTAAAAGGTAACTAGAATTATTCTTTCTCATTTTCAAAAGTGATTT
TTGCATTATTAAATTTCCCTGCCATTGTAATGCCATTTCACGCAGAAAAAAAGTCA
GCCAGTAATTAAGAAAAAAAGTGATGGAGATTAAGTAGTATTTTGGCTTATTTTTA
GGACTCATCATGAGAAGACACAGTTCCTTTAATCAGGAAATTAATATCCATAATTT
TCACTCAAAATTGCAGTATGTAAAGCAGATTCTCAAAAACTCTCCTGAACACTTAT
TTATATATATGTTTTTATATAAGTAAAATTTTTCTCATATTTTTATACGATATGCAC
ACACACACATACATGCACATACTACTTACTACATGTTCTGTACTTGTACTTTGTACC
ATGCATATTCAAATGTTTATATACATAAGTTTATTATAACATAAACAGTAAAAGTA
ATGAATACTGTTTAAAATAACTAATATAGTATTTTTTAATTTTTGTGGGGATGGATT
CTCAAATACTTGTGATTTTAAAAGATTCTAAAGCTAAAACACAACTTGATTTTAAA
AAGAATGATTCTCCTTACACAATTATAAATATTTGCAGTAAATATTTTCCTTATAAT
ACTGTTTTGACCCCATTTAAAAAGTATTAGATTATATTCCTTTGATCCAATGAAAAC
TGAACCTTATAAATGGTTAGCTGAAAGTAGACCTTATTCTTGTCCTTCTTTAGAAG
AGTAAAGATTTGTCCTAGGGAAGATGGCTGACTTCGGTTCCCAACATGCGTATGCA
TTTAGACTGTAGCTCCTCAGCCCTGTGGACACAAAATTTGGACAGCTTATTAGGTT
ACGTTAGCAATGCATGACGGTTTCTCCAACACTAAGATATTCACGTTGAAACAGAT
TTCCTGTTCGTCTTATGTGTCTGGTAAAATTGTTTCCCCAATTACAATTTGACATAT
CAATAGAGGGTTAACAAGAGTATAATTACATAACAGAATTCCTCATGAACTGTAA
TCAGTCTACAGGAAAATCATTATTTTATCTTGATTTGCAGATGAATATACTGCTAA
GAAAGGGAGCAACTCTGACCTTTGTTAAAGTTGATCTTTTGTAATTGAGGTATAAG
GTATGAAAAGATAAAAAACCGAAGGCCAGAGAATCAGGAAATGAAAGATAGTAT
GGACTGAAGGTAACAATATTTTAATGTTATGCAATATAGTCAGAGAAATATTAAA
AATTAGTTGTTTGCTGTGCATAGGTGGATCTCGCAGGAAGCTAATGAAACCTAAGC
TTCAGTGCCTCTCACTTAGACATGTTCCATTCGAGGTCCTGAACCTAACTTTGTATT
AGGAATTCTGTACTAATTTTGTTGAAGAAGACCAGCAAAGTTGTGTACACTTCTAC
CCCCACAAAATCTGCATTGTCCATGTGAGTAAAGTAAAATAATTCCTGTTATTTTTT
TCTGTTAGAAATAAGTATGGAGGATATGTTTTTAAAAATTTATGAGTTAATTGAAA
TATCCATATATAACAAGTGACTTTCTCACAATATATATGATGTGATATATAGGGAG
ATAGTTTCACTTTCATCATATTTTATACGTTGATTCTGAACTATAGAAAAATAATAA
ATGGGATTTTAATTATAGCTCTTAGTTGGGAAAGAAATATAGAGAGATGTGGGATT
TGAATGCCCATGAAAGACATTTTATTTTACTTGAATATATTCTTGCTTCACTTTACC
CTCCATAATATGTTGTACATTAGTGCTGATCAAGTTTACAGAGTTACATTTTGCTTT
CCTAACCATTCAGTCAGGAATTAAAATATGGCATTGTATAACAACTGGGAAGAAG
CTCATAGTGGATATAAATTAGAGTAGATAATGGGTCACCTTGATAGCCTCTGTTTA
CATTACTTGTATATGGGCAAAATAATTATTACCTATACGTGTATTTAAGCTTAATTT
TCATATAAACAGTATTTTTAATCTATGTTAAAATAGATAATATCTAAAAGTGTGAT
CTCTAGGTAGTCCTTAGTTTATTAGTACTGTACTTCAAAAAGATTTTTAAATAGGTC
CGGCACGGTGGCTCATGCCTGTAATCCCAGCACTTTGGGAGGCTGAGGCGGGCGA
ATCACCTGAGGTCAGGAGTTCGAGATCAGCCTGGCCAACATGGTGAAACCCTGTCT
CAACTAAAAATATAAAAATTAGCCGGGCGTGGTGGCAGGCGCCTGTAATCCCAGC
TACTCGGGAGGCTGAGGCAGGAGAATCACTTGAACCCAAGGGGCAGAAGCTGCAG
TTAGCCAAGATCGCATCATTGCACTCCAGCCTAGGGGACAAGAGCGCGAGACTTC
ATCTCAAAAAAAAAAAAAAAAAAAAAAAAAGATTTTTAAATAATAGCTAAAGGT
ATGCTCTCTAGGTCATCCTTAGTTTATTAGTACTGTACTTAAAAATTATTTTTTAAT
AGTCAATTTTGGGAGATAATTATTTCTTTCCTTATATTTTCCAATTAGTTGGTGTCT
AAAAATAAATGTTTTGTCTAATTTTAGATCAGGTATACATTCACAAAAGCATAAAT
CATAGTCTCACAGGAAATTCACCAATTTTCCATATGTCGTGAGATAACTGTCCTTTC
TACAACCTCATAACAATGAATTTATATAATTACCTAGATTTTCTTAGTGTGAATCTA
CCCATTAGTTTTATTTTCTTGGTAGTTATTTTTTTCCCTCCTCTCTGTTACTATTGGC
CTTAAAATACACAGAGGACGGTTACAGTGTCCTAATAGCTGTTACATGTGTGTGTT
TCAGCGTACTTGAATCAAGTGTACATTTATAGTACCAATAACCGCCTTTACAGCTT
TACAGTTAACAATTCTCTCACAAAACTGTAGAGCATTAGGCATCTGAGAGCCATAG
AGGGCCAACTTTGTTCCAGAGTGAACATGCTTTTTTTCCTCAACATATACACTACTG
ATTTTTTTTAAAAGTATGACTTTCAAGTGAATTAATGTATTGGTTAGGAGAACTGCT
TGCTAAGTCCTTATTACCTCTTGTTAAAGCCTCAGAAGGCCGTGCTGAAAGCCAGA
GGGGAAAAAAAGAGTAATGCACAGGTATCTCTTTTGCAGTGGTGACTGTATTTTGA
GTACCTTGTGTGACAGGGTATTATTACAGCATCTTGTGGGAAAACCTATTAGGCCT
TTGCATGTTAAAGCTGTATAATTTGTTGGGTTGTGAGTGGTCTGACTTAAATGTGTA
TTATAAAATTTAGACATCAAATTTTCCTACTAACTAACTTTATTAGATGCATACTTG
GAAGCACAGTCATATCACACTGGGAGGCAATGCAATGTGGTTACCTGGTCCTAGG
TTTGAACTGTCTTATTTCAAAAGATTTCTGAATTAATTTTTCCCTAGAATTTCTCCTT
CATTCCAAAGTACAAACATACTTTGAAGAATGAAACAGATTGTTCCCATGAATGTA
TGCTCATACTCGACTAGAAACGATCTATGTTAAATGACTGTGTATATGAATTATTT
CAAGTACTACCCCAAATAACTTTCTTATTGCTCTGAAAGAAGAAAAGCAATGTAAA
TCACTATGATTATTGCACAAACAACCAGAATTCTCCAACAATTTTAAGTAATCTGA
TCCTCTTCTTGGAGAAAATTGTTACCTAATAGTTTTTCCTTATGAATGTTATTACTA
CTGGTATAAATCAAATTTCTATAAATTTCCTACTTAAGTCTTAAGAACTGGGTTCTT
CCTTTGATGTTATTCATGTTCAGAAAGGAAACAACACTTTACTCTTTTAGGACAATT
CCTAGAATCTATAGTAGTATCAGGATATATTTTGCTTTAAAATATATTTTGGTTATT
TTGAATACAGACATTGGCTCCAAATTTTCATCTTTGCACAATAGTATGACTTTTCAC
TAGAACTTCTCAACATTTGGGAACTTTGCAAATATGAGCATCATATGTGTTAAGGC
TGTATCATTTAATGCTATGAGATACATTGTTTTCTCCCTATGCCAAACAGGTGAAC
AAACGTAGTTGTTTTTTACTGATACTAAATGTTGGCTACCTGTGATTTTATAGTATG
CACATGTCAGAAAAAGGCAAGACAAATGGCCTCTTGTACTGAATACTTCGGCAAA
CTTATTGGGTCTTCATTTTCTGACAGACAGGATTTGACTCAATATTTGTAGAGCTTG
CGTAGAATGGATTACATGGTAGTGATGCACTGGTAGAAATGGTTTTTAGTTATTGA
CTCAGAATTCATCTCAGGATGAATCTTTTATGTCTTTTTATTGTAAGCATATCTGAA
TTTACTTTATAAAGATGGTTTTAGAAAGCTTTGTCTAAAAATTTGGCCTAGGAATG
GTAACTTCATTTTCAGTTGCCAAGGGGTAGAAAAATAATATGTGTGTTGTTATGTT
TATGTTAACATATTATTAGGTACTATCTATGAATGTATTTAAATATTTTTCATATTC
TGTGACAAGCATTTATAATTTGCAACAAGTGGAGTCCATTTAGCCCAGTGGGAAAG
TCTTGGAACTCAGGTTACCCTTGAAGGATATGCTGGCAGCCATCTCTTTGATCTGT
GCTTAAACTGTAATTTATAGACCAGCTAAATCCCTAACTTGGATCTGGAATGCATT
AGTTATGACCTTGTACCATTCCCAGAATTTCAGGGGCATCGTGGGTTTGGTCTAGT
GATTGAAAACACAAGAACAGAGAGATCCAGCTGAAAAAGAGTGATCCTCAATATC
CTAACTAACTGGTCCTCAACTCAAGCAGAGTTTCTTCACTCTGGCACTGTGATCAT
GAAACTTAGTAGAGGGGATTGTGTGTATTTTATACAAATTTAATACAATGTCTTAC
ATTGATAAAATTCTTAAAGAGCAAAACTGCATTTTATTTCTGCATCCACATTCCAA
TCATATTAGAACTAAGATATTTATCTATGAAGATATAAATGGTGCAGAGAGACTTT
CATCTGTGGATTGCGTTGTTTCTTAGGGTTCCTAGCACTGATGCCTGCACAAGCAT
GTGATATGTGAAATAAAATGGATTCTTCTATAGCTAAATGAGTTCCCTCTGGGGAG
AGTTCTGGTACTGCAATCACAATGCCAGATGGTGTTTATGGGCTATTTGTGTAAGT
AAGTGGTAAGATGCTATGAAGTAAGTGTGTTTGTTTTCATCTTATGGAAACTCTTG
ATGCATGTGCTTTTGTATGGAATAAATTTTGGTGCAATATGATGTCATTCAACTTTG
CATTGAATTGAATTTTGGTTGTATTTATATGTATTATACCTGTCACGCTTCTAGTTG
CTTCAACCATTTTATAACCATTTTTGTACATATTTTACTTGAAAATATTTTAAATGG
AAATTTAAATAAACATTTGATAGTTTACATAA
Homo sapiens cysteine/glutamate transporter SLC7A11 protein, Accession No.
NP_055146.1 (SEQ ID NO: 4):
MVRKPVVSTISKGGYLQGNVNGRLPSLGNKEPPGQEKVQLKRKVTLLRGVSIIIGTIIG
AGIFISPKGVLQNTGSVGMSLTIWTVCGVLSLFGALSYAELGTTIKKSGGHYTYILEVF
GPLPAFVRVWVELLIIRPAATAVISLAFGRYILEPFFIQCEIPELAIKLITAVGITVVMVLN
SMSVSWSARIQIFLTFCKLTAILIIIVPGVMQLIKGQTQNFKDAFSGRDSSITRLPLAFYY
GMYAYAGWFYLNFVTEEVENPEKTIPLAICISMAIVTIGYVLTNVAYFTTINAEELLLS
NAVAVTFSERLLGNFSLAVPIFVALSCFGSMNGGVFAVSRLFYVASREGHLPEILSMIH
VRKHTPLPAVIVLHPLTMIMLFSGDLDSLLNFLSFARWLFIGLAVAGLIYLRYKCPDMH
RPFKVPLFIPALFSFTCLFMVALSLYSDPFSTGIGFVITLTGVPAYYLFIIWDKKPRWFRI
MSEKITRTLQIILEVVPEEDKL

Exemplary human FAM3B mRNA and protein sequences are:

Homo sapiens FAM3 metabolism regulating signaling
molecule (FAM3B) transcript variant 1, mRNA,
Accession No. NM_058186.4 (SEQ ID NO: 5)
CTTCCTGACCCAGGGGCTCCGCTGGCTGCGGTCGCCTGGGAGCTGCCGCC
AGGGCCAGGAGGGGAGCGGCACCTGGAAGATGCGCCCATTGGCTGGTGGC
CTGCTCAAGGTGGTGTTCGTGGTCTTCGCCTCCTTGTGTGCCTGGTATTC
GGGGTACCTGCTCGCAGAGCTCATTCCAGATGCACCCCTGTCCAGTGCTG
CCTATAGCATCCGCAGCATCGGGGAGAGGCCTGTCCTCAAAGCTCCAGTC
CCCAAAAGGCAAAAATGTGACCACTGGACTCCCTGCCCATCTGACACCTA
TGCCTACAGGTTACTCAGCGGAGGTGGCAGAAGCAAGTACGCCAAAATCT
GCTTTGAGGATAACCTACTTATGGGAGAACAGCTGGGAAATGTTGCCAGA
GGAATAAACATTGCCATTGTCAACTATGTAACTGGGAATGTGACAGCAAC
ACGATGTTTTGATATGTATGAAGGTGATAACTCTGGACCGATGACAAAGT
TTATTCAGAGTGCTGCTCCAAAATCCCTGCTCTTCATGGTGACCTATGAC
GACGGAAGCACAAGACTGAATAACGATGCCAAGAATGCCATAGAAGCACT
TGGAAGTAAAGAAATCAGGAACATGAAATTCAGGTCTAGCTGGGTATTTA
TTGCAGCAAAAGGCTTGGAACTCCCTTCCGAAATTCAGAGAGAAAAGATC
AACCACTCTGATGCTAAGAACAACAGATATTCTGGCTGGCCTGCAGAGAT
CCAGATAGAAGGCTGCATACCCAAAGAACGAAGCTGACACTGCAGGGTCC
TGAGTAAATGTGTTCTGTATAAACAAATGCAGCTGGAATCGCTCAAGAAT
CTTATTTTTCTAAATCCAACAGCCCATATTTGATGAGTATTTTGGGTTTG
TTGTAAACCAATGAACATTTGCTAGTTGTATCAAATCTTGGTACGCAGTA
TTTTTATACCAGTATTTTATGTAGTGAAGATGTCAATTAGCAGGAAACTA
AAATGAATGGAAATTCTTAAAGGGAATGATGTGATTCAAGCTGGAAAGAG
GGTTGGGAGAAACAGCTTGTCCAGGTGGAGCTATGTTATGATCAGATCGA
AGTGTGACCCCTGTGTGGTCCAGACAGCCCTGCAGAGAGAAAACCTTTAT
TCCATTATCACCAAGCACCTCCTAGTTTCCGACAGTCATCTCCTTCTGCT
GGGAGAATTAGCAGCAGTTCAGGGGGCTTATGTTATGTCCTTGTTCAACT
CAACTTGAGCTCTTGAACTCCTCCTGTGGGCCTGTGAATGTATTCATTCA
TTCCACAACTCTGGGTG
Homo sapiens FAM3 metabolism regulating signaling
molecule (FAM3B) protein, Accession No. NP_055146.
1 (SEQ ID NO: 6):
MRPLAGGLLKVVFVVFASLCAWYSGYLLAELIPDAPLSSAAYSIRSIGER
PVLKAPVPKRQKCDHWTPCPSDTYAYRLLSGGGRSKYAKICFEDNLLMGE
QLGNVARGINIAIVNYVTGNVTATRCFDMYEGDNSGPMTKFIQSAAPKSL
LFMVTYDDGSTRLNNDAKNAIEALGSKEIRNMKFRSSWVFIAAKGLELPS
EIQREKINHSDAKNNRYSGWPAEIQIEGCIPKERS

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the instant disclosure, the therapeutically effective dose can be estimated initially from cell culture assays. A dose may be formulated in animal models to achieve a circulating plasma concentration range that includes the IC50 (i.e., the concentration of Telaglenastat, or a metabolite or derivative thereof, which achieves a half-maximal inhibition of symptoms and/or a half-maximal extent of killing of targeted cancer cells) as determined in cell culture. Such information can be used to more accurately determine useful doses in humans. Levels in plasma may be measured, for example, by high performance liquid chromatography.

Telaglenastat has the following structure:

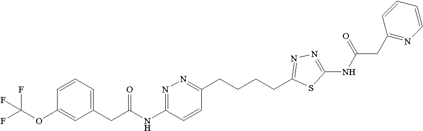 [SMILES: O=C(Cc1cccc(OC(F)(F)F)c1)Nc1ccc(CCCCc2nnc(NC(=O)Cc3ccccn3)s2)nn1]

Exemplary Telaglenastat dose regimens include, but are not limited to the following, for advanced/metastatic tumors:

600 mg tablet taken twice daily on 28 day cycles in combination with talazoparib. The talazoparib may be given as a 1 mg tablet taken daily on 28 day cycles in combination with Telaglenastat. (NCT03875313) 600 mg tablet taken twice daily on 28 day cycles in combination with palbociclib. The palbociclib may be taken as a 75 mg tablet taken on days 1-21 of every 28 day cycle in combination with Telaglenastat.

### Combination Treatments

The compositions and methods of the present disclosure may be used in the context of a number of therapeutic or prophylactic applications. In order to increase the effectiveness of a treatment with the compositions of the present disclosure, e.g., a glutaminase inhibitor including but not limited to Telaglenastat, BPTES, Glutaminase-IN-1, 968, or derivatives thereof, selected and/or administered as a single agent, or to augment the efficacy of another therapy (second therapy), it may be desirable to combine these compositions and methods with one another, or with other agents and methods effective in the treatment, amelioration, or prevention of diseases and pathologic conditions.

In certain embodiments of the instant disclosure, one or more chemotherapeutic drugs that are unrelated to glutaminase inhibitors can be co-administered with a glutaminase inhibitor, or can be administered in advance of glutaminase inhibitor administration. Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Pharmaceutical Compositions

Agents of the present disclosure can be incorporated into a variety of formulations for therapeutic use (e.g., by administration) or in the manufacture of a medicament (e.g., for treating or preventing cancer, e.g., a cancer expresses high levels of SLC7A11 and/or low levels of SLC25A45 and/or FAM3B) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences. Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made wider GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as a glutaminase inhibitor, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic. i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid. L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press. Boca Raton, Fla., 1987, pp 137-149

### Pharmaceutical Dosages

Pharmaceutical compositions of the present disclosure containing an agent described herein may be used (e.g., a glutaminase inhibitor) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W “The Use of Interspecies Scaling in Toxicokinetics,” In Toxicokinetics and New Drug Development, Yacobi et al., Eds. Pergamon Press. New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such as magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g a glutaminase inhibitor) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for oral delivery or intravenous injection to a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a glutaminase inhibitor, e.g., Telaglenastat, a Telaglenastat derivative, BPTES, a BPTES derivative, Glutaminase-IN-1 or a Glutaminase-IN-1 derivative) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a glutaminase inhibitor) described herein. In certain embodiments, a dose described herein includes independently between 1 mg and 3 mg, inclusive, of an agent (e.g., a glutaminase inhibitor) described herein. In certain embodiments, a dose described herein includes independently between 3 mg and 10 mg, inclusive, of an agent (e.g., a glutaminase inhibitor) described herein. In certain embodiments, a dose described herein includes independently between 10 mg and 30 mg, inclusive, of an agent (e.g., a glutaminase inhibitor) described herein. In certain embodiments, a dose described herein includes independently between 30 mg and 100 mg, inclusive, of an agent (e.g., a glutaminase inhibitor) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g. a glutaminase inhibitor) or composition, as described herein, can be administered in combination with one or more additional pharmaceutical agents (e.g., therapeutically and/or prophylactically active agents), which are different from the agent or composition and may be useful as, e.g., combination therapies.

The agents or compositions can be administered in combination with additional pharmaceutical agents that improve their activity (e.g., activity (e.g., potency and/or efficacy) in treating a disease (e.g., cancer) in a subject in need thereof, in preventing a disease in a subject in need thereof, in reducing the risk of developing a disease in a subject in need thereof, etc. in a subject or cell. In certain embodiments, a pharmaceutical composition described herein including an agent e.g a glutaminase inhibitor described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an immunotherapy, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, glutaminase inhibitors, other anti-cancer agents, immunotherapy and/or immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may, be essentially continuous over a preselected period of time or may be in a series of spaced doses.

Guidance regarding particular dosages and methods of delivery is provided in the literature; see, for example, U.S. Pat. Nos. 4,657,760; 5,206,344, or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a glutaminase inhibitor) of this disclosure and/or may contain agents (e.g, oligonucleotide primers, probes, etc.) for identifying a cancer or subject as chemotherapeutic resistant and/or as exhibiting elevated SLC7A11 and/or reduced SLC25A45 and/or FAM3B levels. In some embodiments, the kits further include instructions for use in accordance with the methods of this disclosure. In some embodiments, these instructions comprise a description of administration of the agent to treat or diagnose, e.g., a cancer that exhibits elevated expression of SLC7A11 and/or reduced expression of SLC25A45 and/or FAM3B and/or amplification of the SLC7A11 locus or mutation of the SLC25A45 and/or FAM3B locus, according to any of the methods of this disclosure. In some embodiments, the instructions comprise a description of how to detect a cancer or subject as chemotherapeutic resistant and/or as exhibiting elevated SLC7A11 and/or reduced SLC25A45 and/or FAM3B, for example in an individual, in a tissue sample, or in a cell. The kit may further comprise a description of selecting an individual suitable for treatment based on identifying whether that subject has a cancer that is chemotherapeutic resistant and/or as exhibits elevated SLC7A11 and/or reduced SLC25A45 and/or FAM3B.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating. e.g., a cancer or subject as chemotherapeutic drug resistant and/or as exhibiting elevated SLC7A11 or reduced SLC25A45 and/or FAM3B mRNA or protein levels, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent in the composition is a glutaminase inhibitor. The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### PRISM Screening

Parental cell lines were obtained from the Cancer Cell Line Encyclopedia (CCLE) project. PRISM cell line barcoding, pooling, and screening was performed as previously described with several improvements to the original method. First, the lentiviral vector was modified to encode the unique barcode identifier at the end of the puromycin resistance gene. This enables barcodes to be detected using a variant of the mRNA capture and Luminex detection method developed for the L1000 gene expression assay. Second, a set of ten inert barcodes were spiked-in to each well of each plate after cell lysis to control for variation in PCR amplification as detailed below.

### Data Processing

Luminex technology produced .Ixb files containing data for each Luminex bead observed during detection. These .Ixb files were processed to compute Median Fluorescence Intensity (MFI) values, calculated as the median of the values obtained for all beads corresponding to a single PRISM barcode.

MFI values were log-transformed (log MFI) and used to perform basic quality control. To detect probable screening artifacts, log MFI values were centered to the median log MFI for each cell line on each plate in order to put the measurements from each cell line on the same scale. For each well on each plate, the median of these centered values was then standardized according to the global median and global MAD across all plate wells in the same position. Data from wells with a standardized score of greater than 5 or less than −5 were excluded from all further processing steps.

For each cell line on each plate, the distribution of MFI values observed for the DMSO-treated negative controls was compared to that of the positive controls using a robust form of the Strictly Standardized Mean Difference (SSMD*)’. Specifically, SSMD* was calculated as:

$$\frac{( {µ}_{-} - {µ}_{+} )}{\sqrt{{σ}_{-}^{2} + {σ}_{+}^{2}}}$$

Data corresponding to SSMD values less than 2 were removed before calculating cell viability.

The data in the instant disclosure were produced according to two different screening protocols. In the PR500 protocol, ten inert barcodes were spiked-in to each well of each plate after cell lysis. For data produced using the PR500 protocol, normalized MFI (nMFI) values were computed by taking the ratio of each MFI value against the median of the inert barcodes within each well. For data produced before the PR500 protocol was introduced, nMFI values were set equal to MFI values.

Cell viability was calculated as the ratio of nMFI to the median of the nMFI from the DMSO-treated negative controls for each cell line on each plate. Batch effects produced from variable detection and assay conditions were then removed using ComBat (Johnson et al. Biostatistics 8: 118-127). The final viability values were calculated as the median of the batch-corrected cell viabilities from biological replicates for each cell line, compound and dose.

### Dose Response

Measures of dose response were obtained by fitting 3-parameter logistic curves to viability values for each compound and cell line using the R package ‘drc’. Following the practice of Smirnov and Safikhani (Smirnov et al. Bioinformatics 32: 1244-1246), viability was truncated at 1.0 and fit as a function of drug concentration according to:

$$V ( c ) = {E}_{∞} + \frac{1 - {E}_{∞}}{1 + {e}^{HS ( c - EC 50 )}}$$

where all concentrations are in the natural logarithm scale. IC50 values were defined as the concentration c at which V(c)=0.5, given by the formula:

$$IC 50 = - \frac{log ( 1 - 2 {E}_{∞} )}{HS} + EC 50$$

The Area Under the dose response Curve (AUC) was calculated using the normalized integral:

$$\frac{{\int }_{{c}_{min}}^{{c}_{max}} V ( c ) d c}{{c}_{max} - {c}_{min}}$$

where

$$\int V ( c ) d c = \frac{( {E}_{∞} - 1 ) log ( 1 + {e}^{- HS ( c - EC 50 )} )}{HS} + {E}_{∞} c + const$$

The formulation above puts AUC values on a scale between 0 and 1, where lower AUC values indicate increased sensitivity to the treatment.

RNAseq gene expression data was obtained from the CCLE website (portals.broadinstitute.org/ccle). Identity of all human cell lines was confirmed by STR fingerprinting (Genetica).

### CellTiter-Glo® Cellular Viability Assay

Cell viability was assayed using a modified manufacturer's protocol for CellTiter-Glo® (Promega #G7573). Cells were seeded at a density of 2000 cells per well in a 96 well black, clear bottom plate (Corning #89091-012) in 100 uL total media per well. The following day different concentrations of compounds at various doses were printed in triplicate in a random well format using the Tecan D300e Digital Dispenser. After 72 hours, 60 μL of a 1:3 solution of CellTiterGlo reagent in 1×PBS (Corning #01018002) was added per well and allowed to incubate at RT for 10 mins. Luminescence was measured with an integration time of 0.1 s using Envision Microplates Reader (PERKIN ELMER #2105-0010). Biological replicates were averaged and normalized to vehicle control. Dose curves were generated using Graphpad Prism.

### Example 2: High-Throughput Cytotoxicity Profiling of Telaglenastat Revealed SLC25A45 and SLC7A11 as Glutaminase-Inhibitor Sensitive Biomarkers in Cancer Cells

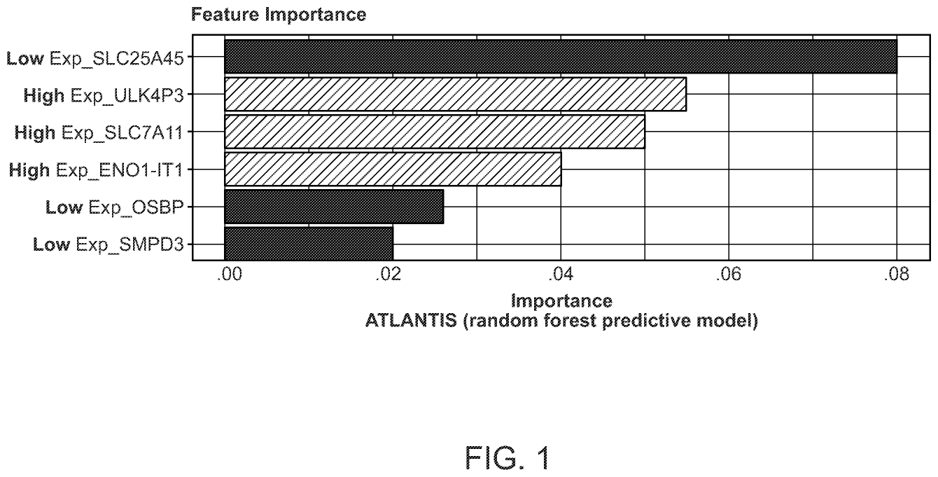

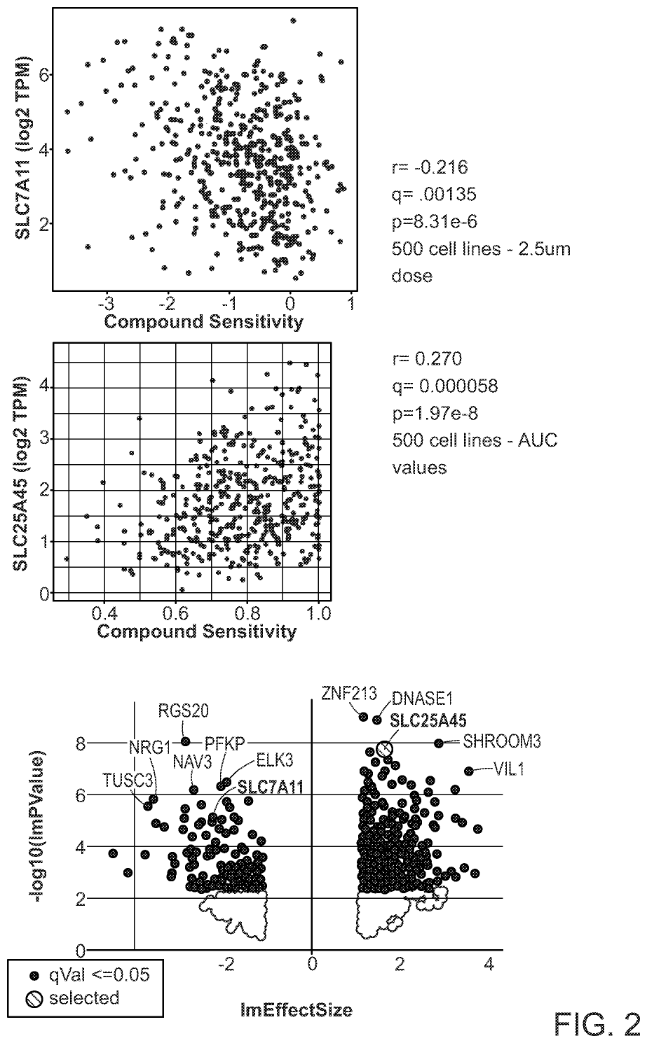

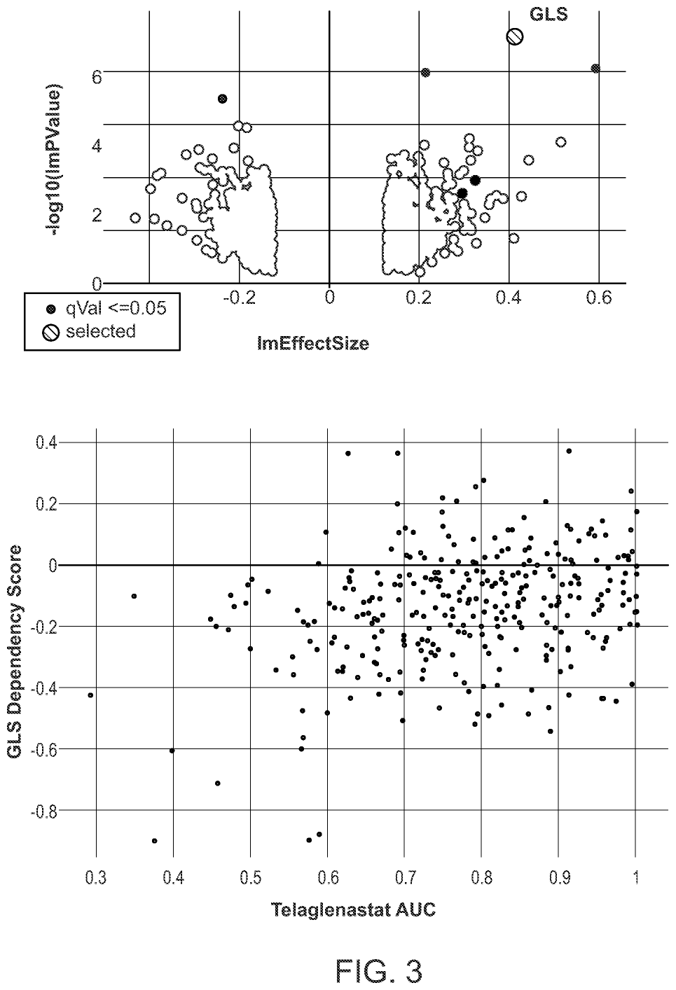

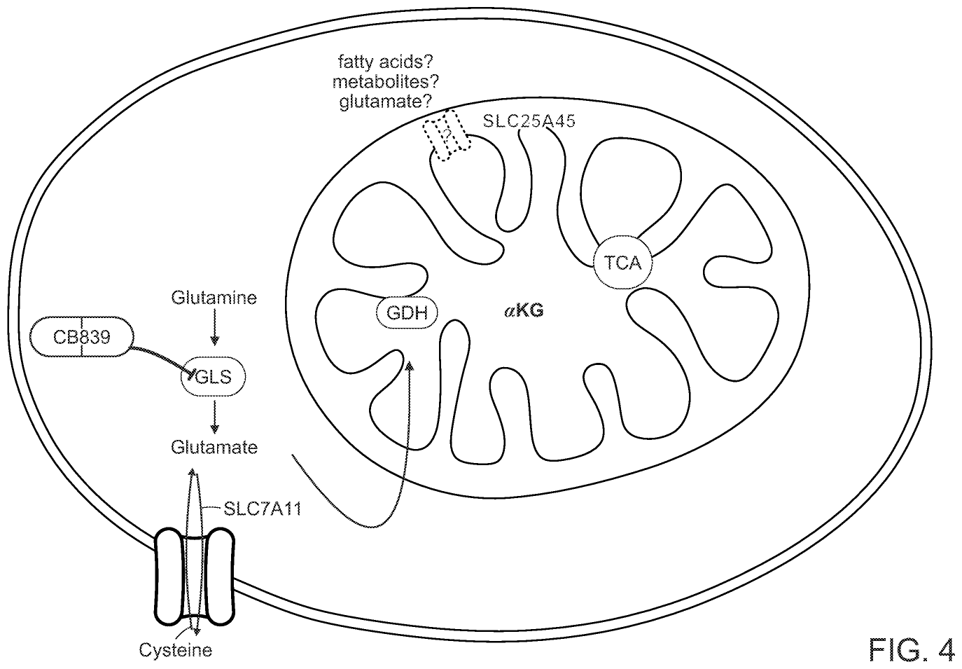

A bar graph of the PRISM screen predictions employed herein to identify Telaglenastat biomarkers is presented in FIG. 1. In 500 adherent cell lines, multivariate models concluded that the top two important features for predicting Telaglenastat tumor cell line sensitivity were low expression of SLC25A45 and high expression of SLC7A11. In the plot of FIG. 1, green depicts lower expression associated with killing and red depicts higher expression associated with killing. For biomarker analysis, multiple ATLANTIS predictive models were trained for the Telaglenastat PRISM profile. Cross-validated R square values and Pearson scores were then reported, with feature importance results also determined. As shown in FIG. 2, high SLC7A11 expression and low SLC25A45 expression were discovered to correlate with Telaglenastat tumor cell killing in PRISM. Using Pearson correlations and associated p-values, univariate analysis between Telaglenastat sensitivity and genomic features revealed significant associations between SLC7A11 (top panel) and SLC25A45 (middle panel) expression levels and Telaglenastat-mediated cell-line killing. The Benjamin Hochberg algorithm was used to compute q values from p values. Compound sensitivity has been defined herein as log 2 fold change in viability compared to DMSO treatment. AUC values from MTS011 500 adherent cell lines were depicted as volcano plot data (bottom panel). As shown in FIG. 3, glutaminase (GLS) knockout was the top-correlated CRISPR feature in the Telaglenastat PRISM profile, which was indicative of Telaglenastat on-target activity. The volcano plot presented in FIG. 3 (top panel) depicts the positive association observed between Telaglenastat AUC values and CRISPR knockout scores. The bottom panel of FIG. 3 also demonstrates that Telaglenastat-sensitive cell line survival was highly dependent on GLS.

Given the role for glutaminase inhibitors identified herein, a schematic of the biochemical mechanisms by which glutaminase inhibitors have been modeled to starve sensitive cells is depicted in FIG. 4. Low levels of SLC25A45 fatty acid transporter reduce the level of acetylCoA entering the Krebs/tricarboxylic acid cycle (TCA). Further, high levels of SLC7A11 glutamate/cysteine transporter reduce the level of glutamate converted to alpha-ketoglutarate. Thus, without wishing to be bound by theory, both low expression of SLC25A45 and high expression of SLC7A11, as have been identified as biomarkers herein, likely result in lower input levels of metabolites into the Krebs/tricarboxylic acid cycle (TCA), thereby lowering available ATP, which is believed to be the mechanism by which cell death is potentiated.

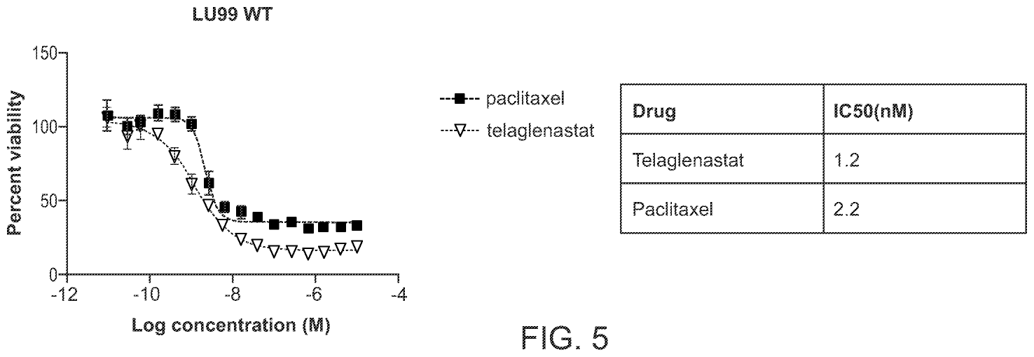

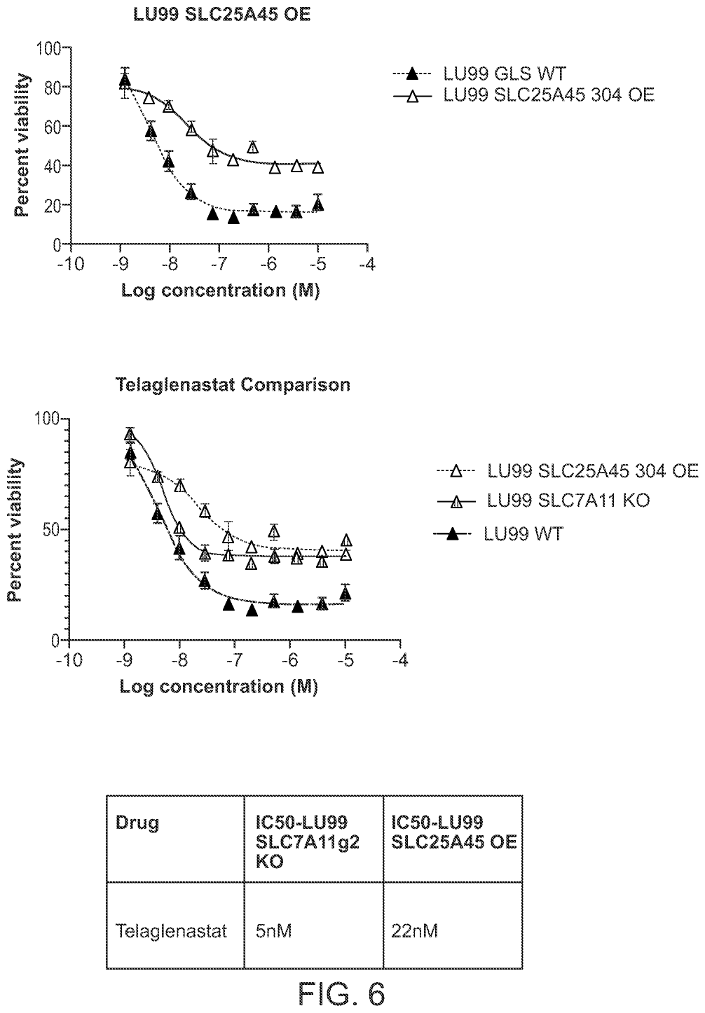

As shown in FIG. 5, LU99 cells (human lung giant cell carcinoma cell line) were identified to exhibit high sensitivity to Telaglenastat. The percent viability of LU99 cells observed herein has been plotted in FIG. 5 vs. the log concentration of Telaglenastat and Paclitaxel. The IC50 of Telaglenastat (1.2 nM) was on the order of that of Paclitaxel, a potent anti-mitotic drug (2.2 nM). As shown in FIG. 6, SLC25A45 overexpression increased LU99 cell viability in response to Telaglenastat treatment and raised the Telagelenastat IC50 to 22 nM.

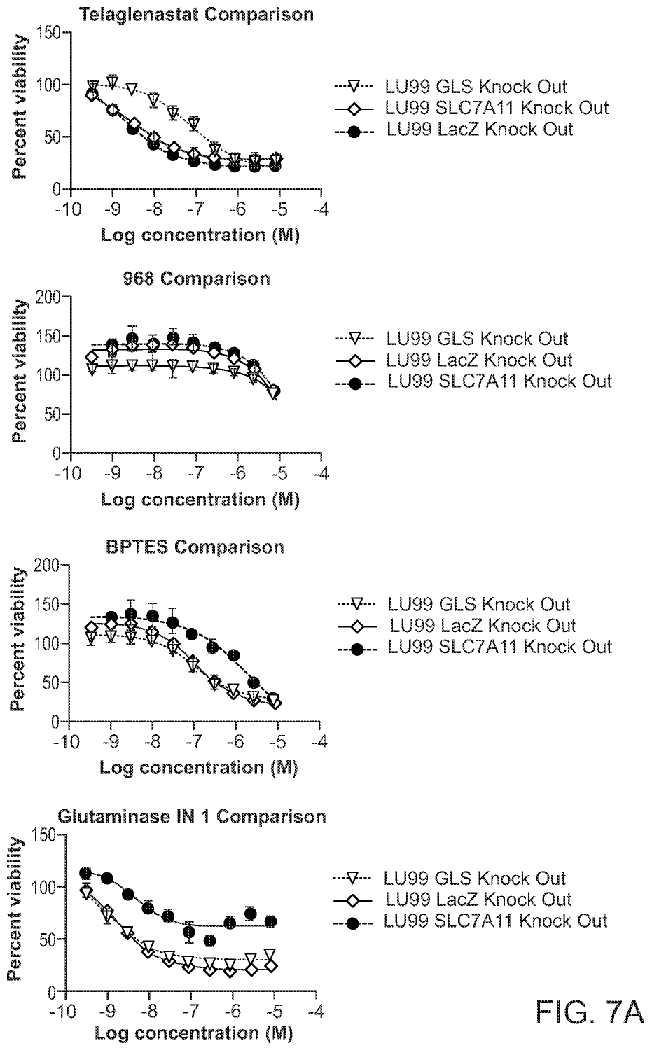

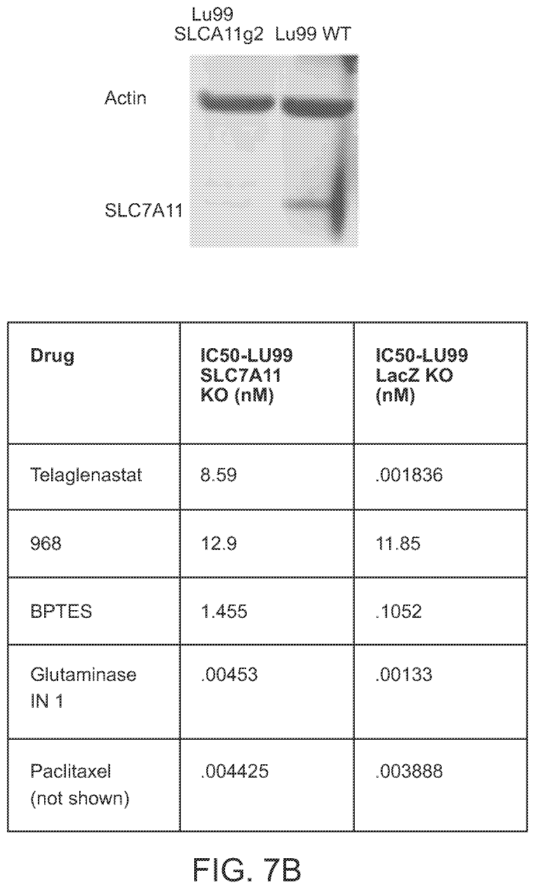

As shown in FIGS. 6, 7A, and 7B, knock-out of SLC7A11 in LU99 cells rescued cells from Telaglenastat-induced cell killing. While LU99 cells exhibited sensitivity to multiple GLS inhibitors, sensitivity of LU99 cells to 968 was not rescued by knock-out of SLC7A11, which, without wishing to be bound by theory, indicated that inhibition of the KGA isoform (NCBI identifiers: NP_055720.3, NM_014905.4, uniprot identifier: 094925-1) rather than the GAC isoform (NCBI identifiers: NP_001243239.1, NM_001256310.1, uniprot identifier: 094925-3) of glutaminase was likely necessary for 968-induced cell killing. Plots of glutaminase inhibitor cell killing and attempted rescue results observed for LU99 cells harboring knock-outs of glutaminase, SLC7A11, or LacZ (control) vs glutaminase inhibitor log concentration are shown in FIG. 7A (Telaglenastat at top; 968 in second panel; BPTES in third panel; and Glutaminase IN 1 at bottom). As shown in FIG. 7B by western blot, efficient knock-out of SLC7A11 in LU99 cells was confirmed. Meanwhile, the IC50 values observed for the various glutaminase inhibitors in LU99 cells, both in response to SLC7A11 knock-out vs LacZ knock-out (control) are also tabulated in FIGS. 6 and 7B.

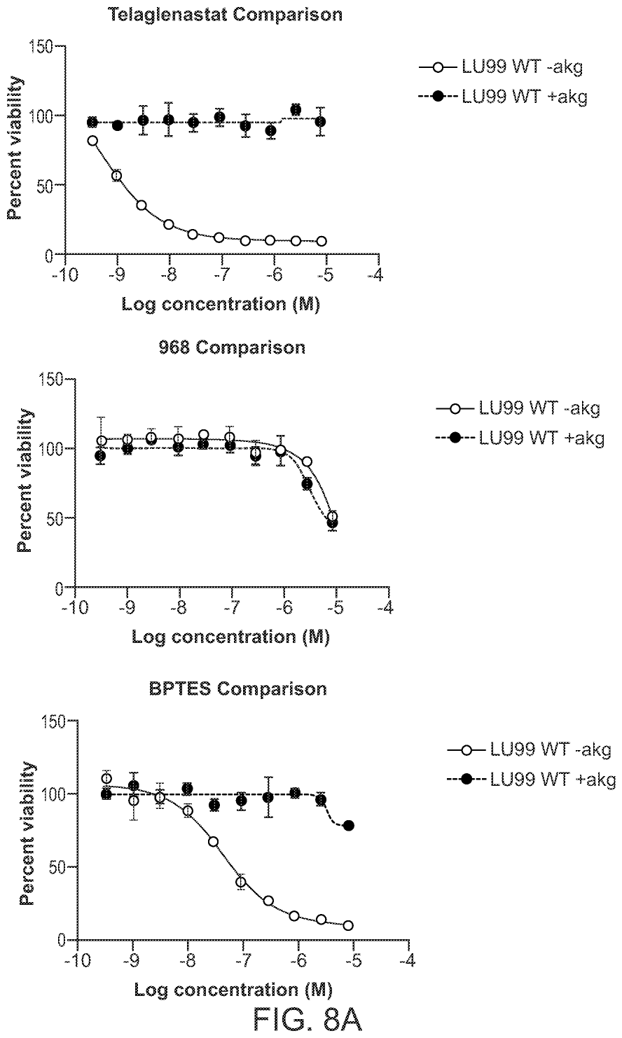

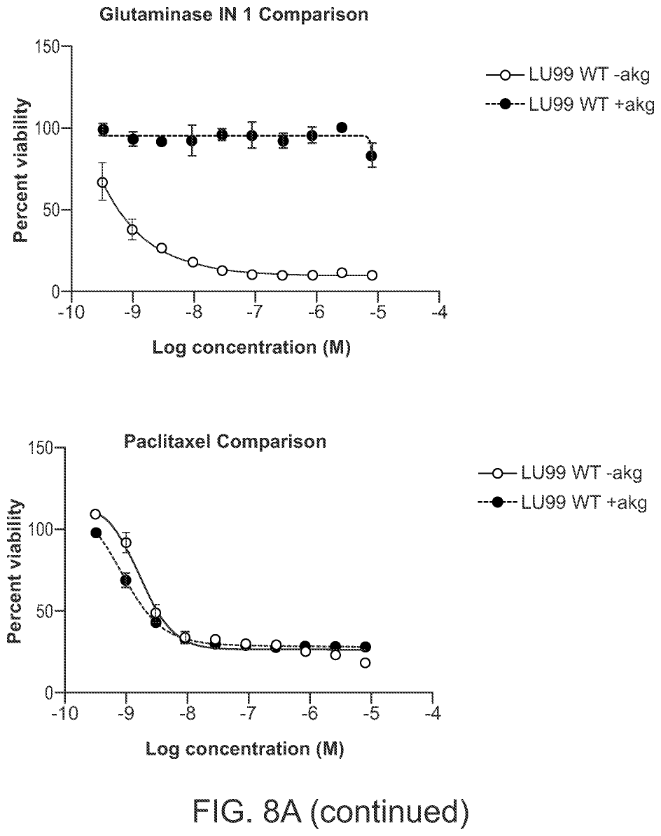

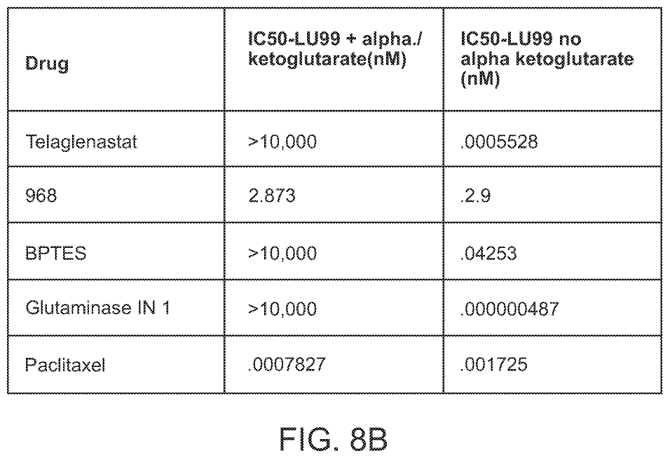

As shown in FIGS. 8A and 8B, addition of alpha ketoglutarate to LU99 cell media also rescued cells from Telaglenastat-induced cell death. FIG. 8A shows the percent viability of LU99 cells administered indicated glutaminase inhibitors (showing response vs the log concentration (M) of glutaminase inhibitor), in the presence and absence of 2 mM alpha ketoglutarate. The five plots of FIG. 8A show results for Telaglenastat, 968, BPTES, Glutaminase IN 1, and Paclitaxel, respectively. Tabulated IC50 values (FIG. 8B) show that, apart from 968, the IC50 of each glutaminase inhibitor increased by many orders of magnitude with alpha ketoglutarate in the media. Without wishing to be bound by theory, these data demonstrated that glutaminase inhibitors induce cell death via starvation.

### Example 3: SLC7A11 Knockout and SLC25A45 Overexpression Also Protected NCI-H2122 Cells from Telaglenastat and Other Glutaminase Inhibitors

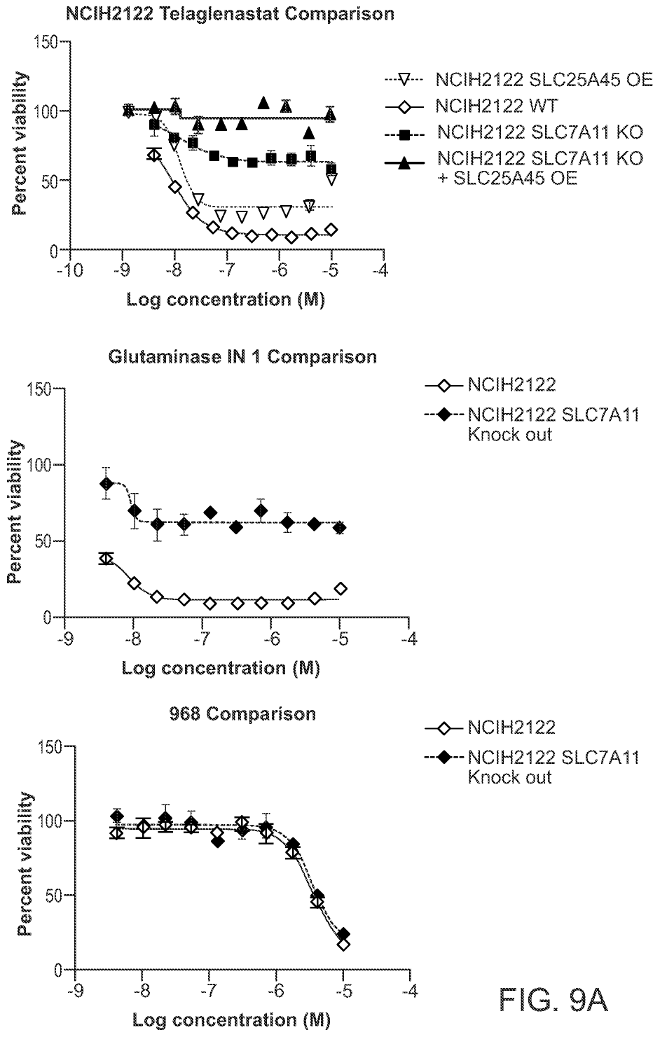

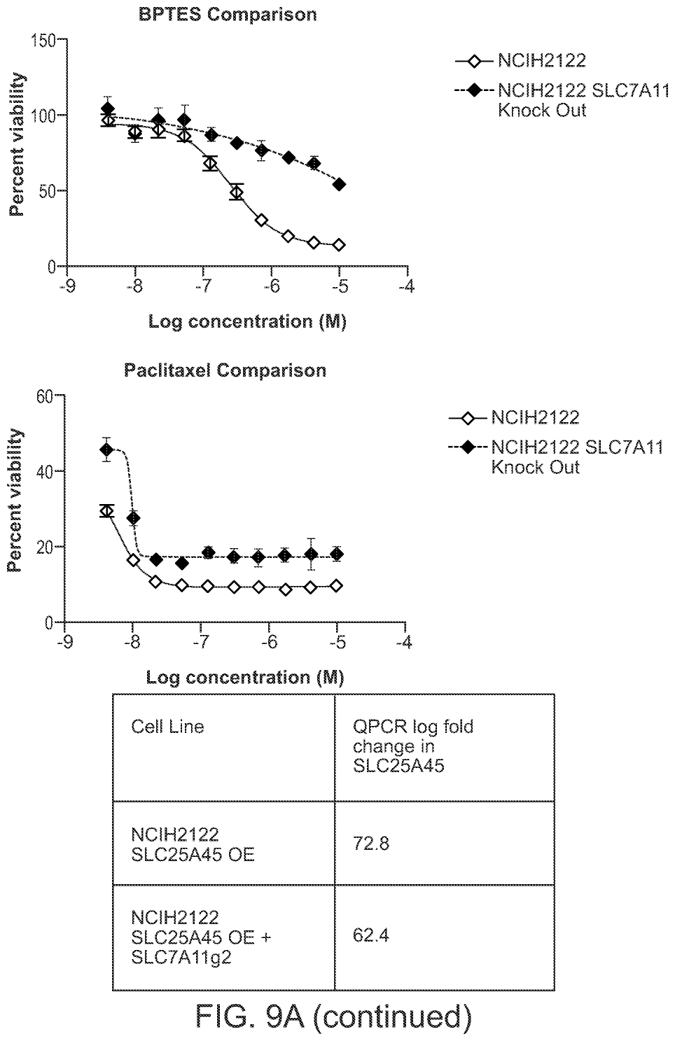

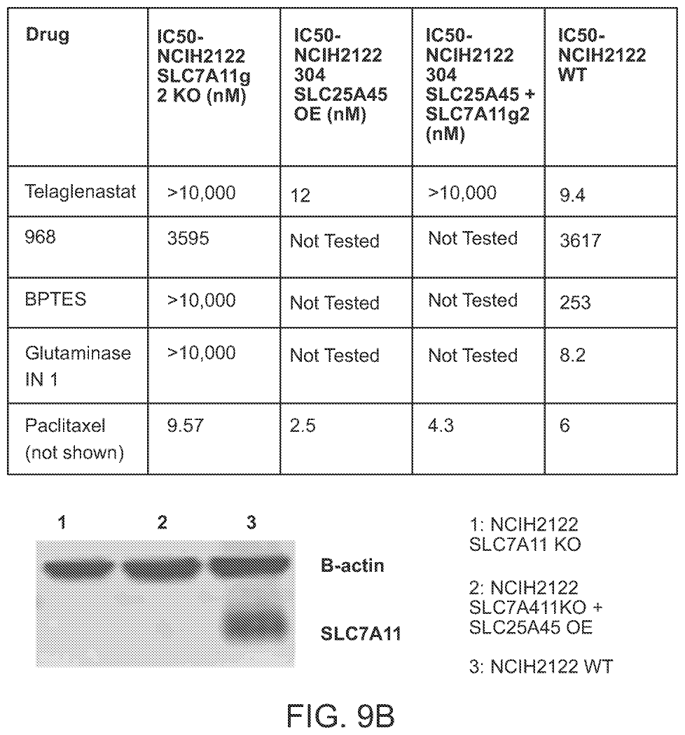

The impacts of SLC7A11 knock-out and SLC25A45 over-expression were also examined in another highly Telaglenastat-sensitive cell line, NCI-H2122 (human lymphoblast non-small cell lung cancer cells). In NCI-H2122 cells, both SLC7A11 knock-out and SLC25A45 over-expression were identified to increase cell viability in response to treatment with the following glutaminase inhibitors: Telaglenastat, Glutaminase IN 1, and BPTES (FIGS. 9A and 9B). Meanwhile, no significant rescue of sensitivity was observed in the current experiments for the 968 glutaminase inhibitor, nor for Paclitaxel (a microtubule-stabilizing drug).

### Example 4: Genome-Side CRISPR Screen of LU99 Cells Confirmed SLC7A11 Knockout as a Top Hit for Rescue from Telaglenastat Sensitivity

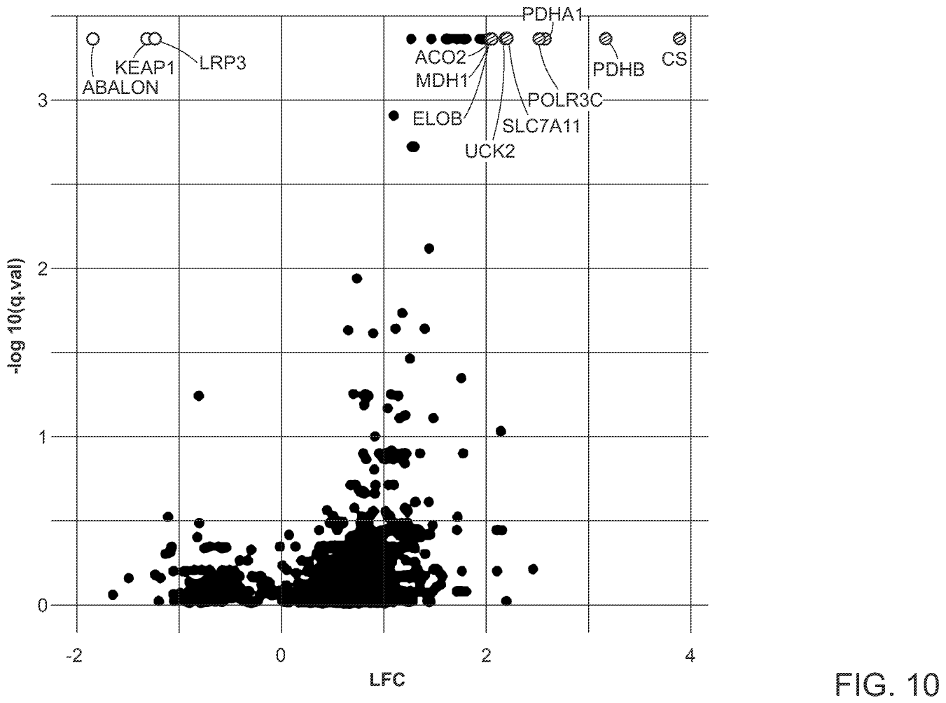

A genome-wide CRISPR knockout screen was also performed upon LU99 cells treated with Telagenastat for 9 days, with observation of cell viability (cell death). Statistically significant sgRNA-mediated knock-out results were observed for SLC7A11 sgRNA (FIG. 10), with a high LFC in cell death observed. This result validated the above-described findings that SLC7A11 expression is critical in mediating Telaglenastat sensitivity in the LU99 cell line.

### Example 5: Ectopic Expression of FAM3B Also Rescued LU99 Cells from Telaglenastat Sensitivity

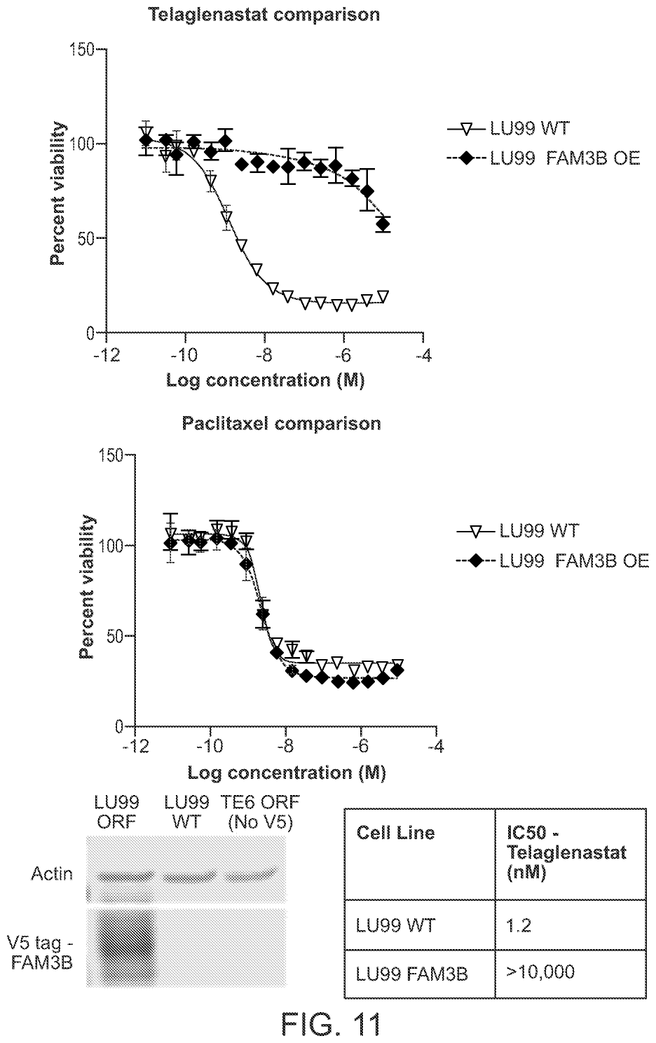

Notably, ectopic expression of FAM3B was also found to exhibit significant Telaglenastat rescue capability in LU99 cells. In particular, ectopic expression of FAM3B in LU99 cells increased the Telaglenastat IC50 to greater than 10,000 nM (relative to 1.2 nM for Telaglenastat treatment of wild type LU99 cells; FIG. 11). Moreover, this effect appeared to be specific to Telaglenastat as compared to Pacliataxel, as ectopic FAM3B expression induced no change in Pacliataxel effects on cell death (FIG. 11). FAM3B was therefore identified as acting with similar effect and directionality as SLC25A45.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for selecting a treatment for a subject having or at risk of developing a cancer, the method comprising:
(a) providing or having provided a sample from a subject having or at risk of developing a cancer;
(b) identifying or having identified said sample as glutaminase inhibitor-sensitive if one or more of the following is observed in the sample:
(i) elevated levels of SLC7A11 mRNA or protein, as compared to an appropriate control and/or amplification of the SLC7A11 locus;
(ii) reduced levels of SLC25A45 mRNA or protein, as compared to an appropriate control and/or mutation of the SLC25A45 locus, as compared to an appropriate control,
(iii) reduced levels of FAM3B mRNA or protein, as compared to an appropriate control and/or mutation of the FAM3B locus, as compared to an appropriate control,
thereby identifying said sample as glutaminase inhibitor-sensitive; and
(c) selecting a glutaminase inhibitor for treatment of the subject so identified as providing a glutaminase inhibitor-sensitive sample, thereby selecting a treatment for said subject.

2. The method of claim 1, wherein the cancer is a solid tumor or a hematopoietic cancer, optionally wherein the cancer is a kidney, lung, pancreas, esophageal, or gastric cancer and/or wherein the cancer is an advanced and/or metastatic cancer.

3. The method of claim 1, wherein step (b) comprises identifying or having identified the presence in the sample of elevated SLC7A11 mRNA levels and reduced SLC25A45 and/or FAM3B mRNA levels, as compared to an appropriate control.

4. The method of claim 1, wherein the mutation of the SLC25A45 locus disrupts SLC25A45 mRNA or protein function.

5. The method of claim 1, wherein the mutation of the FAM3B locus disrupts FAM3B mRNA or protein function.

6. The method of claim 1, wherein the KGA isoform of glutaminase is inhibited, optionally wherein the glutaminase inhibitor is selected from the group consisting of Telaglenastat (CB-839), BPTES (Bis-2-(5-phenylacetamido-1,3,4-thiadiazol-2-yl)ethyl sulfide) and Glutaminase-IN-1, a pharmaceutically acceptable salt, ester, amide, prodrug or stereoisomer thereof, or a derivative thereof.

7. The method of claim 1, further comprising: (d) administering or having administered the selected glutaminase inhibitor to the subject.

8. The method of claim 1, further comprising selecting a non-glutaminase inhibitor chemotherapeutic drug as a treatment for the subject.

9. The method of claim 1, wherein identifying step (b) comprises use of a kit of any one of claims 13-17.

10. The method of claim 1, wherein the subject is human.

11. A method selected from the group consisting of:
A method for treating or preventing cancer in a subject, comprising:
(a) providing or having provided a sample from a subject having or at risk of developing cancer;
(b) identifying or having identified the sample as glutaminase inhibitor-sensitive if one or more of the following is observed in the sample:
(i) elevated levels of SLC7A11 mRNA or protein, as compared to an appropriate control and/or amplification of the SLC7A11 locus;
(ii) reduced levels of SLC25A45 mRNA or protein, as compared to an appropriate control and/or mutation of the SLC25A45 locus, as compared to an appropriate control,
(iii) reduced levels of FAM3B mRNA or protein, as compared to an appropriate control and/or mutation of the FAM3B locus, as compared to an appropriate control,
thereby identifying or having identified the sample as glutaminase inhibitor-sensitive; and
(c) administering or having administered a glutaminase inhibitor to the subject, thereby treating or preventing cancer in the subject; and
A method for treating a subject having a cancer that is resistant to a non-glutaminase inhibitor chemotherapeutic drug, the method comprising identifying one or more of the following in the cancer of the subject:
(i) elevated levels of SLC7A11 mRNA or protein, as compared to an appropriate control and/or amplification of the SLC7A11 locus;
(ii) reduced levels of SLC25A45 mRNA or protein, as compared to an appropriate control and/or mutation of the SLC25A45 locus, as compared to an appropriate control;
(iii) reduced levels of FAM3B mRNA or protein, as compared to an appropriate control and/or mutation of the FAM3B locus, as compared to an appropriate control; and
administering or having administered to the subject a glutaminase inhibitor, thereby treating the subject having a cancer that is resistant to a non-glutaminase inhibitor chemotherapeutic drug.

12. (canceled)

13. A composition selected from the group consisting of:
A kit for identifying elevated expression of SLC7A11 mRNA or protein in a sample consisting essentially of an oligonucleotide for detection of SLC7A11 mRNA or an anti-SLC7A11 antibody, optionally wherein the anti-SLC7A11 antibody is labeled or wherein the kit comprises a labeled secondary antibody that binds the anti-SLC7A11 antibody, and instructions for its use;
A kit for identifying reduced expression of SLC25A45 or FAM3B mRNA or protein in a sample consisting essentially of an oligonucleotide for detection of SLC25A45 or FAM3B mRNA or an anti-SLC25A45 or anti-FAM3B antibody, optionally wherein the anti-SLC25A45 or anti-FAM3B antibody is labeled or wherein the kit comprises a labeled secondary antibody that binds the anti-SLC25A45 or anti-FAM3B antibody, and instructions for its use;
A kit for identifying elevated expression of SLC7A11 mRNA or protein and reduced expression of SLC25A45 and/or FAM3B mRNA or protein in a sample, the kit consisting essentially of: (1) an oligonucleotide for detection of SLC7A11 mRNA or an anti-SLC7A11 antibody, optionally wherein the anti-SLC7A11 antibody is labeled or wherein the kit comprises a labeled secondary antibody that binds the anti-SLC7A11 antibody; and (2) an oligonucleotide for detection of a SLC25A45 mRNA or a FAM3B mRNA, or an anti-SLC25A45 or an anti-FAM3B antibody, optionally wherein the anti-SLC25A45 or anti-FAM3B antibody is labeled or wherein the kit comprises a labeled secondary antibody that binds the anti-SLC25A45 or anti-FAM3B antibody, and instructions for its use; and
A pharmaceutical composition for treating a subject having a cancer that exhibits one or more of the following:
(1) elevated expression of SLC7A11 mRNA or protein, as compared to an appropriate control and/or amplification of the SLC7A11 locus; and/or
(2) reduced levels of SLC25A45 mRNA or protein, as compared to an appropriate control and/or mutation of the SLC25A45 locus, as compared to an appropriate control,
(3) reduced levels of FAM3B mRNA or protein, as compared to an appropriate control and/or mutation of the FAM3B locus, as compared to an appropriate control,
the pharmaceutical composition comprising a therapeutically effective amount of a glutaminase inhibitor and a pharmaceutically acceptable carrier.

14-15. (canceled)

16. The kit of claim 13, wherein the sample is a cancer sample, optionally wherein the cancer is a solid tumor or hematopoietic cancer, optionally wherein the cancer is a kidney, lung, pancreas, esophageal, or gastric cancer and/or wherein the cancer is an advanced and/or metastatic cancer.

17. The kit of claim 13, wherein the sample is a tissue sample of a subject having a solid tumor or a hematopoietic cancer, optionally wherein the cancer is a kidney, lung, pancreas, esophageal, or gastric cancer and/or wherein the cancer is an advanced and/or metastatic cancer.

18. (canceled)

19. The pharmaceutical composition of claim 13, wherein the cancer is a solid tumor or hematopoietic cancer, optionally wherein the cancer is a kidney, lung, pancreas, esophageal, or gastric cancer and/or wherein the cancer is an advanced and/or metastatic cancer.

20. The pharmaceutical composition of claim 13, wherein the KGA isoform of glutaminase is inhibited, optionally wherein the glutaminase inhibitor is selected from the group consisting of Telaglenastat, BPTES and Glutaminase-IN-1, a pharmaceutically acceptable salt, ester, amide, prodrug or stereoisomer thereof, or a derivative thereof.

21. The pharmaceutical composition of claim 13, wherein the subject is human.
